(19) United States
(12) Patent Application Publication
(10) Pub. No.: US 2012/0315454 A1
(43) Pub. Date: Dec. 13, 2012

(54) NATURAL BIOPOLYMER THERMOPLASTIC FILMS

(76) Inventors: James H. Wang, Appleton, WI (US); Yan Wang, Beijing (CN); Xueen George Hao, Beijing (CN); Tongtong Zhang, Beijing (CN)

(21) Appl. No.: 13/510,775

(22) PCT Filed: Dec. 1, 2010
(86) PCT No.: PCT/IB10/55533

§ 371 Date: May 18, 2012

(30) Foreign Application Priority Data
Dec. 31, 2009 (CN) 200910260747.X

(51) Int. Cl. C08L 67/00; B32B 7/00; B29C 47/14; C08L 23/06; C08L 33/06; C08K 5/1545; B32B 27/32; A61L 15/22

(52) U.S. Cl. 428/220; 428/500; 604/372; 524/52; 524/53; 264/177.1

(57) ABSTRACT

A thermoplastic film composition that includes a polymer blend of multiple inherently incompatible polymer components is described. The composition includes a polymer blend having about 5 wt. % to about 45 wt. % of a plasticized natural polymer, about 5 wt. % to about 40 wt % of a polyolefin, a biodegradable polymer, and a compatibilizer with both a polar and a non-polar moiety on the same polymer molecule. The total plasticized natural and biodegradable polymers constitute a majority or predominant phase (≧51 wt. %), while petroleum-based olefinic polymers form the minority phase. The composition can be made into a film containing at least one renewable, natural polymer component. Also described are the articles of manufacture that may use such films.

### FIELD OF INVENTION

The present invention relates to a thermoplastic film composition. In particular, the invention describes a polymer blend of multiple inherently incompatible polymer components in a film and the uses of the resultant film. The films contain at least one renewable, natural polymer component.

### BACKGROUND

As the general public develops a wider social awareness of so-called “green” technologies and a desire to purchase products made from renewable materials, manufacturers are facing a challenge to try to respond to this consumer demand. Moreover, governmental requirements increasingly mandating the use of renewable or reusable materials in certain classes of disposable products has spurred a need to develop better and more innovative ways to deal with waste. In recent years manufacturers of plastic or thermoplastic products or materials have shown increasing interest in cellulose or starch-based materials as an important, environmentally friendly natural resource. As a kind of biodegradable biopolymer, starch is one of the most abundant natural polymers that can be renewably produced each year in large quantities. Manufacturers are seeking new ways to incorporate more recyclable or natural and biodegradable materials into otherwise conventional polymer-based products.

Natural polymers are produced in nature by absorbing carbon dioxide, a green house gas responsible for global warming. The materials containing natural biopolymers will have reduced environmental foot print in terms of the overall energy savings, reduction of green house gas emission, etc. throughout the life cycle of the products, including raw material productions, manufacturing, distribution, use, end-of-life disposal, etc.

In particular, there is an increased business need to develop biomaterial-based and biodegradable thin films for use in the field of absorbent articles, such as infant and child care products, feminine hygiene products, and adult incontinence products, etc. For instance, these films can be incorporated as outercover films in diapers and training pants, adult incontinence articles or garments, and baffle films for feminine pantiliners, pads and incontinence pads. None of the current commercially available biomaterial-based and biodegradable materials alone meet the application needs of such products. Conventional polylactic acid (PLA) is too rigid for quiet flexible film applications and tends to have performance in use issues, such as causing noisy rustles for adult feminine products. Aliphatic-aromatic copolyester films, such as Ecoflex® films are synthetic polymer films made from petroleum and do not contain any natural or biomaterial-based polymer component needed for the intended application and their costs are also too high for such intended applications. Pure copolyester also exhibits poor converting processability for fabricating cast films. The resultant film is too sticky and cannot be collected by winding up on a roll. The copolyester cast film also tends to block easily making it very difficult, if not impossible, to separate into individual layers after it is produced. Typically copolyester is used in polymer blends with other polymers to overcome the above deficiencies. Thermoplastic starch (TPS) alone cannot be made into thin films due to limited processability, the resulting films from pure thermoplastic starch are also very brittle and rigid to be useful for soft flexible film applications. Films made from blends of thermoplastic starch and copolyesters can be made into soft thin films, and the material costs are too expensive for the intended applications.

In view of these difficulties and shortcomings of currently available materials, an unmet need exists in the thin films for personal care product applications. It is highly desirable to invent relatively inexpensive polymer blend formulations that can be used to create soft and malleable thermoplastic cast film that contains a significant amount of naturally-derived biodegradable components.

### SUMMARY OF THE INVENTION

The present invention relates, in part, to a formulation for polymer blended composition that contain a majority of biodegradable content, which can be employed to make thin cast films. The inventive compositions are engineered polymer blends of multiple inherently incompatible polymer components. The compositions include: a plasticized natural polymer such as a thermoplastic starch, thermoplastic plant protein, or microbial polyester-polyhydroxyalkanoate (PHA), a biodegradable polymer such as a copolyester (e.g. Ecoflex), a polyolefin (e.g., polyethylene), and a compatibilizer that has both a polar and a non-polar moiety on the same polymer (e.g. maleic anhydride, acrylic acid, hydroxyethyl methacrylate, glycidyl (meth)acrylate, etc. grafted polyolefins). The total amount of biodegradable components constitutes a majority phase (>50 wt. %) of the dry polymer blend. In typical embodiments the biodegradable contents at least 53 wt. %, or can be from about 55-60 wt. % up to about 70-80 wt. % or 85 wt. %. The amount by weight of polyolefins may range from about 5% to about 40%, plasticized natural polymers from about 5% to about 45%, biodegradable polymer (i.e. copolyester) from about 5% to about 75%, and compatibilizer from about 0.5% to about 15%. Additional components also may be included in the composition are pigments (e.g., TiO₂), antioxidants, slip additives, and anti-blocking agents, etc, up to about 5 wt. % or 6 wt. % total. The resulting thin cast films can be made into baffle film for various adult incontinence care and feminine care products; outercover films for diapers, training pants, swim pants products; packaging films, that are biomaterial-based and mostly biodegradable. Hence, the invention also pertains to absorbent articles that incorporate parts made with the present polymer blend. Another embodiment of this invention is a blown film made from the inventive compositions which can be used as packaging film, outer cover films for absorbent products, or baffle films for absorbent products.

In another aspect, the present invention describes a method of producing the polymer blend system to fabricate cast thermoplastic films. The method involves blending the multiple polymer components in one or more melt extrusion steps, either separately or simultaneously extruding thin films from the polymer compositions. In one embodiment, thermoplastic natural polymer is produced in a separate step which involves the plasticization of the natural polymers by melt blending with one or more plasticizers.

### BRIEF DESCRIPTION OF FIGURES

FIGS. 1A and 1B are a schematic representation of polymeric and biodegradable components within a cast thermoplastic film. FIG. 1A illustrates the relative amounts of polymeric and biodegradable components in a conventional film sample, and FIG. 1B illustrates the relative amounts of each according to a film embodiment of the present invention.

FIG. 2 is a SEM image of a cross-section of a film made according to an embodiment of the present invention.

FIG. 3 is a SEM image of a cross-section of a film made according to another embodiment of the present invention.

### DETAILED DESCRIPTION OF THE INVENTION

### Section I

### DEFINITION

The term “biodegradable,” as used herein, refers generally to a material that can degrade from the action of naturally occurring microorganisms, such as bacteria, fungi, yeasts, and algae; environmental heat, moisture, or other environmental factors. If desired, the extent of biodegradability may be determined according to ASTM Test Method 5338.92.

The term “renewable” as used herein refers to a material that can be produced or is derivable from a natural source which is periodically (e.g., annually or perennially) replenished through the actions of plants of terrestrial, aquatic or oceanic ecosystems (e.g., agricultural crops, edible and non-edible grasses, forest products, seaweed, or algae), or microorganisms (e.g., bacteria, fungi, or yeast).

### Section II

### DESCRIPTION

The present invention arises from technical development to engineer a biodegradable complex, multi-component polymer blend system, which contains chemically incompatible components, the resulting polymer blend has a majority of biodegradable polymer contents. The polymer blend system is characterized by novel and synergistic interactions. As a collective system, through the innovative formation and interaction of an olefinic polymer compatibilized polymer microstructure and morphology, a finely dispersed polymer system is created to exhibit the combined desired attributes and features of good polymer processability, biodegradability, and mechanical strength performance needed for applications in the intended disposable product market, even though each polymer component individually may not exhibit the proper or required properties and processability attributes.

Although binary and tertiary polymer blend systems have been developed before, such as the blends of TPS/Ecoflex, PE/TPS/compatibilizer, etc., these kinds of resulting blends either lack the desired processability or are too costly for disposable products uses. It is believed that a four-component polymer blend system with the properties and good processability is not obvious to those skilled in the art. Further, the present invention involves creating a polymer blend system from what had been considered to be mutually incompatible ingredients for producing a film having the desired characteristics and properties. Extensive control systems were also developed to demonstrate the non-obviousness of the invention.

### A. Film Material Components

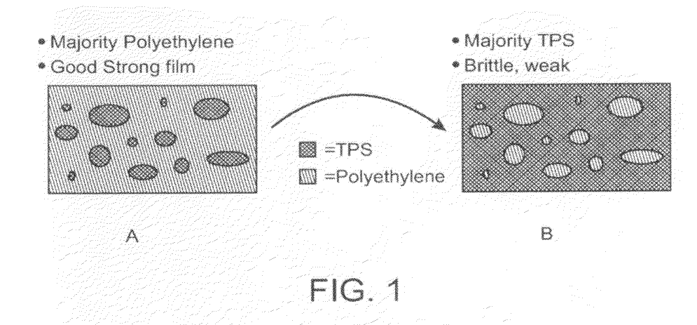

The concept of the present invention, in part, can be explained or illustrated with reference to the schematic representations of FIGS. 1A and 1B, which depicts a change from a polyolefin (PE) majority phase to a TPS (thermoplastic starch) majority phase. FIG. 1A shows a conventional film substrate that is predominately made from a polyolefin (PE) (e.g., polypropylene) with a minority phase of TPS or other materials or fillers. Mechanistically, as in FIG. 1A, when polyolefin is the majority phase, it forms a continuous phase. Since polyolefins have the physical characteristics necessary to form a thin film, the resulting blend could be made into a thin film without any complications. FIG. 1B depicts a film according an objective of the present invention in which plasticized natural and biodegradable polymers constitute the majority or predominant phase, while the petroleum-based olefinic polymers form the minority phase. Previously, efforts of making 60% thermoplastic starch masterbatch and 40% polyolefins have failed to yield a thin film of any utility because the material tended to tear easily, be very brittle, and have low tensile properties. As the amount of biopolymer TPS increases to over 50% in volume (e.g., 53%, 55%, 58%, or 60%), it forms a majority phase, since TPS or TPS masterbatch does not exhibit the same processability characteristics for making good quality films, pure TPS ordinarily cannot be used to form a thin film of 1 to 2 mils and is often very rigid and brittle, the resulting polymer blend lacks the required mechanical properties and ability to be processed into thin films. Since the material processability and properties is commonly determined by the continuous phase (most often the majority phase) of the materials, the two proportionate compositions contribute to a difference in mechanism of making films. A novel approach in composition and processing needs to be developed to overcome these technical challenges, which the present invention addresses.

To overcome this problem, creative blends compositions were surprisingly produced with the addition of the right amount of an additional synthetic biodegradable polymer, an aliphatic-aromatic copolyester to the mix even though the copolyester itself has limitation to form a cast film. The overall components were made compatible by one or more compatibilizers. The resulting films were surprisingly soft, homogeneous, and having balanced mechanical properties desired for the baffle film applications.

The polyolefin and thermoplastic starch molecules are not chemical bonded with each other, nor are starch-polyester graft copolymers included. The polymer blend system is not a water-based suspension. The film casting process does not involve evaporation steps. The starch particles are not crosslinked. It is important to have non-crosslinked starch to form thin films, otherwise the particles are filler and may cause film debonding.

According to the present invention, the natural and biodegradable components constitute a majority phase of the polymer blend, while polyolefins make up the minority phase. The polyolefin content can be from about 5 wt. % to about 45 wt. %, but more typically is in an intermediate range (e.g., about 10-35 wt. %, 15-30 wt. %, 20-40 wt. %, or 22-37 wt %). The theoretic maximum combined amount of plasticized natural polymer and biodegradable polymer can total 100%, but since incorporation of other ingredients is desirable, a practical maximum for these natural and biodegradable components can be up to about 98% of the polymer blend. It is desired that no oxidizing agent is used in the present formulation.

### 1. Biodegradable Polyester

Like those materials described in U.S. Patent Application Publication No. 2008-0147034A1, relating to a water-sensitive biodegradable film, the content of which is incorporated herein by reference, the film of the present invention includes one or more biodegradable polyesters. The biodegradable polyesters employed in the present invention typically have a relatively low glass transition temperature (“T_{g}”) to reduce stiffness of the film and improve the processability of the polymers. For example, the T_{g} may be about 25°C. or less, in some embodiments about 0° C. or less, and in some embodiments, about −10° C. or less. Likewise, the melting point of the biodegradable polyesters is also relatively low to improve the rate of biodegradation. For example, the melting point is typically from about 50° C. to about 180° C., in some embodiments from about 80° C. to about 160° C., and in some embodiments, from about 100° C. to about 140° C. The melting temperature and glass transition temperature may be determined using differential scanning calorimetry (“DSC”) in accordance with ASTM D-3417 as is well known in the art. Such tests may be employed using a THERMAL ANALYST 2910 Differential Scanning Calorimeter (outfitted with a liquid nitrogen cooling accessory) and with a THERMAL ANALYST 2200 (version 8.10) analysis software program, which are available from T.A. Instruments Inc. of New Castle, Del.

The biodegradable polyesters employed in the film of the present invention may also have a number average molecular weight (“Mₙ”) ranging from about 40,000 to about 120,000 grams per mole, in some embodiments from about 50,000 to about 100,000 grams per mole, and in some embodiments, from about 60,000 to about 85,000 grams per mole.

Likewise, the polyesters may also have a weight average molecular weight (“M_{w}”) ranging from about 70,000 to about 240,000 grams per mole, in some embodiments from about 80,000 to about 190,000 grams per mole, and in some embodiments, from about 100,000 to about 150,000 grams per mole. The ratio of the weight average molecular weight to the number average molecular weight (“M_{w}/Mₙ”), i.e., the “polydispersity index”, is also relatively low. For example, the polydispersity index typically ranges from about 1.0 to about 4.0, in some embodiments from about 1.2 to about 3.0, and in some embodiments, from about 1.4 to about 2.0. The weight and number average molecular weights may be determined by methods known to those skilled in the art.

The biodegradable polyesters may also have an apparent viscosity of from about 100 to about 1000 Pascal seconds (Pa·s), in some embodiments from about 200 to about 800 Pa·s, and in some embodiments, from about 300 to about 600 Pa·s, as determined at a temperature of 170° C. and a shear rate of 1000 sec⁻¹. The melt flow index of the biodegradable polyesters may also range from about 0.1 to about 10 grams per 10 minutes, in some embodiments from about 0.5 to about 8 grams per 10 minutes, and in some embodiments, from about 1 to about 5 grams per 10 minutes. The melt flow index is the weight of a polymer (in grams) that may be forced through an extrusion rheometer orifice (0.0825-inch diameter) when subjected to a load of 2160 grams in 10 minutes at a certain temperature (e.g., 190° C.), measured in accordance with ASTM Test Method D1238-E.

Of course, the melt flow index of the biodegradable polyesters will ultimately depend upon the selected film-forming process. For example, when extruded as a cast film, higher melt flow index polymers are typically desired, such as about 4 grams per 10 minutes or more, in some embodiments, from about 5 to about 12 grams per 10 minutes, and in some embodiments, from about 7 to about 9 grams per 10 minutes. Likewise, when formed as a blown film, lower melt flow index polymers are typically desired, such as less than about 12 grams per 10 minutes or less, in some embodiments from about 1 to about 7 grams per 10 minutes, and in some embodiments, from about 2 to about 5 grams per 10 minutes.

Examples of suitable biodegradable polyesters include aliphatic polyesters, such as polycaprolactone, polyesteramides, modified polyethylene terephthalate, polylactic acid (PLA) and its copolymers, terpolymers based on polylactic acid, polyglycolic acid, polyalkylene carbonates (such as polyethylene carbonate), polyhydroxyalkanoates (PHA), poly-3-hydroxybutyrate (PHB), poly-3-hydroxyvalerate (PHV), poly-3-hydroxybutyrate-co-4-hydroybutyrate, poly-3-hydroxybutyrate-co-3-hydroxyvalerate copolymers (PHBV), poly-3-hydroxybutyrate-co-3-hydroxyhexanoate, poly-3-hydroxybutyrate-co-3-hydroxyoctanoate, poly-3-hydroxybutyrate-co-3-hydroxydecanoate, poly-3-hydroxybutyrate-co-3-hydroxyoctadecanoate, and succinate-based aliphatic polymers (e.g., polybutylene succinate, polybutylene succinate adipate, polyethylene succinate, etc.); aromatic polyesters and modified aromatic polyesters; and aliphatic-aromatic copolyesters. In one particular embodiment, the biodegradable polyester is an aliphatic-aromatic copolyester (e.g., block, random, graft, etc.). The aliphatic-aromatic copolyester may be synthesized using any known technique, such as through the condensation polymerization of a polyol in conjunction with aliphatic and aromatic dicarboxylic acids or anhydrides thereof. The polyols may be substituted or unsubstituted, linear or branched, polyols selected from polyols containing 2 to about 12 carbon atoms and polyalkylene ether glycols containing 2 to 8 carbon atoms. Examples of polyols that may be used include, but are not limited to, ethylene glycol, diethylene glycol, propylene glycol, 1,2-propanediol, 1,3-propanediol, 2,2-dimethyl-1,3-propanediol, 1,2-butanediol, 1,3-butanediol, 1,4-butanediol, 1,2-pentanediol, 1,5-pentanediol, 1,6-hexanediol, polyethylene glycol, diethylene glycol, 2,2,4-trimethyl-1,6-hexanediol, thiodiethanol, 1,3-cyclohexanedimethanol, 1,4-cyclohexanedimethanol, 2,2,4,4-tetramethyl-1,3-cyclobutanediol, cyclopentanediol, triethylene glycol, and tetraethylene glycol. Preferred polyols include 1,4-butanediol; 1,3-propanediol; ethylene glycol; 1,6-hexanediol; diethylene glycol; and 1,4-cyclohexanedimethanol.

Representative aliphatic dicarboxylic acids that may be used include substituted or unsubstituted, linear or branched, non-aromatic dicarboxylic acids selected from aliphatic dicarboxylic acids containing 1 to about 10 carbon atoms, and derivatives thereof. Non-limiting examples of aliphatic dicarboxylic acids include malonic, malic, succinic, oxalic, glutaric, adipic, pimelic, azelaic, sebacic, fumaric, 2,2-dimethyl glutaric, suberic, 1,3-cyclopentanedicarboxylic, 1,4-cyclohexanedicarboxylic, 1,3-cyclohexanedicarboxylic, diglycolic, itaconic, maleic, and 2,5-norbornanedicarboxylic. Representative aromatic dicarboxylic acids that may be used include substituted and unsubstituted, linear or branched, aromatic dicarboxylic acids selected from aromatic dicarboxylic acids containing 1 to about 6 carbon atoms, and derivatives thereof. Non-limiting examples of aromatic dicarboxylic acids include terephthalic acid, dimethyl terephthalate, isophthalic acid, dimethyl isophthalate, 2,6-napthalene dicarboxylic acid, dimethyl-2,6-naphthalate, 2,7-naphthalenedicarboxylic acid, dimethyl-2,7-naphthalate, 3,4′-diphenyl ether dicarboxylic acid, dimethyl-3,4′ diphenyl ether dicarboxylate, 4,4′-diphenyl ether dicarboxylic acid, dimethyl-4,4′-diphenyl ether dicarboxylate, 3,4′-diphenyl sulfide dicarboxylic acid, dimethyl-3,4′-diphenyl sulfide dicarboxylate, 4,4′-diphenyl sulfide dicarboxylic acid, dimethyl-4,4′-diphenyl sulfide dicarboxylate, 3,4′-diphenyl sulfone dicarboxylic acid, dimethyl-3,4′-diphenyl sulfone dicarboxylate, 4,4′-diphenyl sulfone dicarboxylic acid, dimethyl-4,4′-diphenyl sulfone dicarboxylate, 3,4′-benzophenonedicarboxylic acid, dimethyl-3,4′-benzophenonedicarboxylate, 4,4′-benzophenonedicarboxylic acid, dimethyl-4,4′-benzophenonedicarboxylate, 1,4-naphthalene dicarboxylic acid, dimethyl-1,4-naphthalate, 4,4′-methylene bis(benzoic acid), dimethyl-4,4′-methylenebis(benzoate), etc., and mixtures thereof.

The polymerization may be catalyzed by a catalyst, such as a titanium-based catalyst (e.g., tetraisopropyltitanate, tetraisopropoxy titanium, dibutoxydiacetoacetoxy titanium, or tetrabutyltitanate). If desired, a diisocyanate chain extender may be reacted with the copolyester to increase its molecular weight. Representative diisocyanates may include toluene 2,4-diisocyanate, toluene 2,6-diisocyanate, 2,4′-diphenylmethane diisocyanate, naphthylene-1,5-diisocyanate, xylylene diisocyanate, hexamethylene diisocyanate (“HMDI”), isophorone diisocyanate and methylenebis(2-isocyanatocyclohexane). Trifunctional isocyanate compounds may also be employed that contain isocyanurate and/or biurea groups with a functionality of not less than three, or to replace the diisocyanate compounds partially by tri- or polyisocyanates. The preferred diisocyanate is hexamethylene diisocyanate. The amount of the chain extender employed is typically from about 0.3 to about 3.5 wt. %, in some embodiments, from about 0.5 to about 2.5 wt. % based on the total weight percent of the polymer.

The copolyesters may either be a linear polymer or a long-chain branched polymer. Long-chain branched polymers are generally prepared by using a low molecular weight branching agent, such as a polyol, polycarboxylic acid, hydroxy acid, and so forth. Representative low molecular weight polyols that may be employed as branching agents include glycerol, trimethylolpropane, trimethylolethane, polyethertriols, 1,2,4-butanetriol, pentaerythritol, 1,2,6-hexanetriol, sorbitol, 1,1,4,4,-tetrakis(hydroxymethyl)cyclohexane, tris(2-hydroxyethyl)isocyanurate, and dipentaerythritol. Representative higher molecular weight polyols (molecular weight of 400 to 3000) that may be used as branching agents include triols derived by condensing alkylene oxides having 2 to 3 carbons, such as ethylene oxide and propylene oxide with polyol initiators. Representative polycarboxylic acids that may be used as branching agents include hemimellitic acid, trimellitic (1,2,4-benzenetricarboxylic) acid and anhydride, trimesic (1,3,5-benzenetricarboxylic) acid, pyromellitic acid and anhydride, benzenetetracarboxylic acid, benzophenone tetracarboxylic acid, 1,1,2,2-ethane-tetracarboxylic acid, 1,1,2-ethanetricarboxylic acid, 1,3,5-pentanetricarboxylic acid, and 1,2,3,4-cyclopentanetetracarboxylic acid. Representative hydroxy acids that may be used as branching agents include malic acid, citric acid, tartaric acid, 3-hydroxyglutaric acid, mucic acid, trihydroxyglutaric acid, 4-carboxyphthalic anhydride, hydroxyisophthalic acid, and 4-(beta-hydroxyethyl)phthalic acid. Such hydroxy acids contain a combination of 3 or more hydroxyl and carboxyl groups. Especially preferred branching agents include trimellitic acid, trimesic acid, pentaerythritol, trimethylol propane and 1,2,4-butanetriol.

The aromatic dicarboxylic acid monomer constituent may be present in the copolyester in an amount of from about 10 mole % to about 40 mole %, in some embodiments from about 15 mole % to about 35 mole %, and in some embodiments, from about 15 mole % to about 30 mole %. The aliphatic dicarboxylic acid monomer constituent may likewise be present in the copolyester in an amount of from about 15 mole % to about 45 mole %, in some embodiments from about 20 mole % to about 40 mole %, and in some embodiments, from about 25 mole % to about 35 mole %. The polyol monomer constituent may also be present in the aliphatic-aromatic copolyester in an amount of from about 30 mole % to about 65 mole %, in some embodiments from about 40 mole % to about 50 mole %, and in some embodiments, from about 45 mole % to about 55 mole %.

In one particular embodiment, for example, the aliphatic-aromatic copolyester may comprise the following structure:

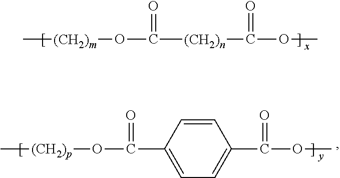

wherein, m is an integer from 2 to 10, in some embodiments from 2 to 4, and in an embodiment, 4;
n is an integer from 0 to 18, in some embodiments from 2 to 4, and in an embodiment, 4;
p is an integer from 2 to 10, in some embodiments from 2 to 4, and in an embodiment, 4;
x is an integer greater than 1; and y is an integer greater than 1.

One example of such a copolyester is polybutylene adipate terephthalate, which is commercially available under the designation ECOFLEX® F BX 7011 from BASF Corp.

Another example of a suitable copolyester containing an aromatic terephtalic acid monomer constituent is available under the designation ENPOL™ 8060M from IRE Chemicals (South Korea). Other suitable aliphatic-aromatic copolyesters may be described in U.S. Pat. Nos. 5,292,783; 5,446,079; 5,559,171; 5,580,911; 5,599,858; 5,817,721; 5,900,322; and 6,258,924, which are incorporated herein in their entirety by reference thereto for all purposes.

Mixtures of two or more aliphatic-aromatic polyesters also could be used, such as described in U.S. Patent Application Publication No. 2009-0157020A1, incorporated herein by reference.

### 2. Thermoplastic Natural Polymers

The thermoplastic natural polymers that can be incorporated in the films of the present invention may include, for instance, thermoplastic starches, other thermoplastic carbohydrate polymers such as thermoplastic cellulose, thermoplastic hemicellulose, thermoplastic lignin derivatives, thermoplastic protein materials (e.g. thermoplastic gluten, thermoplastic soy protein, thermoplastic zein, etc.), thermoplastic algae materials, thermoplastic alginate, etc.

Starch is a natural polymer composed of amylose and amylopectin. Amylose is essentially a linear polymer having a molecular weight in the range of 100,000-500,000, whereas amylopectin is a highly branched polymer having a molecular weight of up to several million. Although starch is produced in many plants, typical sources includes seeds of cereal grains, such as corn, waxy corn, wheat, sorghum, rice, and waxy rice; tubers, such as potatoes; roots, such as tapioca (i.e., cassava and manioc), sweet potato, and arrowroot; and the pith of the sago palm.

Broadly speaking, any natural (unmodified) and/or modified starch may be employed in the present invention. Modified starches, for instance, are often employed that have been chemically modified by typical processes known in the art (e.g., esterification, etherification, oxidation, acid hydrolysis, enzymatic hydrolysis, etc.). Starch ethers and/or esters may be particularly desirable, such as hydroxyalkyl starches, carboxymethyl starches, etc. The hydroxyalkyl group of hydroxylalkyl starches may contain, for instance, 1 to 10 carbon atoms, in some embodiments from 1 to 6 carbon atoms, in some embodiments from 1 to 4 carbon atoms, and in some embodiments, from 2 to 4 carbon atoms. Representative hydroxyalkyl starches such as hydroxyethyl starch, hydroxypropyl starch, hydroxybutyl starch, and derivatives thereof. Starch esters, for instance, may be prepared using a wide variety of anhydrides (e.g., acetic, propionic, butyric, and so forth), organic acids, acid chlorides, or other esterification reagents. The degree of esterification may vary as desired, such as from 1 to 3 ester groups per glucosidic unit of the starch.

A plasticizer is also typically employed in the thermoplastic starch to render the starch melt-processible. Starches normally exist in the form of granules that have a coating or outer membrane that encapsulates the more water-soluble amylose and amylopectin chains within the interior of the granule. When heated, polar solvents (“plasticizers”) may soften and penetrate the outer membrane and cause the inner starch chains to absorb water and swell. This swelling will, at some point, cause the outer shell to rupture and result in an irreversible destructurization of the starch granule. Once destructurized, the starch polymer chains containing amylose and amylopectin polymers, which are initially compressed within the granules, will stretch out and form a generally disordered intermingling of polymer chains. Upon resolidification, however, the chains may reorient themselves to form crystalline or amorphous solids having varying strengths depending on the orientation of the starch polymer chains. Because the starch (natural or modified) is thus capable of melting and resolidifying, it is generally considered a “thermoplastic starch.”

Suitable plasticizers may include, for instance, polyhydric alcohol plasticizers, such as sugars (e.g., glucose, sucrose, fructose, raffinose, maltodextrose, galactose, xylose, maltose, lactose, mannose, and erythrose), sugar alcohols (e.g., erythritol, xylitol, malitol, mannitol, glycerol, and sorbitol), polyols (e.g., ethylene glycol, propylene glycol, dipropylene glycol, butylene glycol, and hexane triol), etc. Also suitable are hydrogen bond forming organic compounds which do not have hydroxyl group, including urea and urea derivatives; anhydrides of sugar alcohols such as sorbitan; animal proteins such as gelatin; vegetable proteins such as sunflower protein, soybean proteins, cotton seed proteins; and mixtures thereof.

Other suitable plasticizers may include phthalate esters, dimethyl and diethylsuccinate and related esters, glycerol triacetate, glycerol mono and diacetates, glycerol mono, di, and tripropionates, butanoates, stearates, lactic acid esters, citric acid esters, adipic acid esters, stearic acid esters, oleic acid esters, and other acid esters. Aliphatic acids may also be used, such as ethylene acrylic acid, ethylene maleic acid, butadiene acrylic acid, butadiene maleic acid, propylene acrylic acid, propylene maleic acid, and other hydrocarbon based acids. A low molecular weight plasticizer is preferred, such as less than about 20,000 g/mol, preferably less than about 5,000 g/mol and more preferably less than about 1,000 g/mol.

The relative amount of starches and plasticizers employed in the thermoplastic starch may vary depending on a variety of factors, such as the molecular weight of the starch, the type of starch (e.g., modified or unmodified), the affinity of the plasticizer for the starch, etc. Typically, however, starches constitute from about 40 wt. % to about 95 wt. %, in some embodiments from about 50 wt. % to about 90 wt. %, and in some embodiments, from about 60 wt. % to about 80 wt. % of the thermoplastic composition. Likewise, plasticizers typically constitute from about 5 wt. % to about 60 wt. %, in some embodiments from about 10 wt. % to about 50 wt. %, and in some embodiments, from about 20 wt. % to about 40 wt. % of the thermoplastic composition. It should be understood that the weight of starch referenced herein includes any bound water that naturally occurs in the starch before mixing it with other components to form the thermoplastic starch. Starches, for instance, typically have a bound water content of about 5% to 16% by weight of the starch.

Other additives may also be employed in the thermoplastic starch to facilitate its use in the film of the present invention. Dispersion aids, for instance, may be employed to help create a uniform dispersion of the starch/plasticizer mixture and retard or prevent separation of the thermoplastic starch into constituent phases. Likewise, the dispersion aids may also improve the water dispersibility of the film. When employed, the dispersion aid(s) typically constitute from about 0.01 wt. % to about 10 wt. %, in some embodiments from about 0.1 wt. % to about 5 wt. %, and in some embodiments, from about 0.5 wt. % to about 4 wt. % of the thermoplastic composition.

Although any dispersion aid may generally be employed in the present invention, surfactants having a certain hydrophilic/lipophilic balance (“HLB”) may improve the long-term stability of the composition. The HLB index is well known in the art and is a scale that measures the balance between the hydrophilic and lipophilic solution tendencies of a compound. The HLB scale ranges from 1 to approximately 50, with the lower numbers representing highly lipophilic tendencies and the higher numbers representing highly hydrophilic tendencies. In some embodiments of the present invention, the HLB value of the surfactants is from about 1 to about 20, in some embodiments from about 1 to about 15 and in some embodiments, from about 2 to about 10. If desired, two or more surfactants may be employed that have HLB values either below or above the desired value, but together have an average HLB value within the desired range.

One particularly suitable class of surfactants for use in the present invention is nonionic surfactants, which typically have a hydrophobic base (e.g., long chain alkyl group or an alkylated aryl group) and a hydrophilic chain (e.g., chain containing ethoxy and/or propoxy moieties). For instance, some suitable nonionic surfactants that may be used include, but are not limited to, ethoxylated alkylphenols, ethoxylated and propoxylated fatty alcohols, polyethylene glycol ethers of methyl glucose, polyethylene glycol ethers of sorbitol, ethylene oxide-propylene oxide block copolymers, ethoxylated esters of fatty (C₈-C₁₈) acids, condensation products of ethylene oxide with long chain amines or amides, condensation products of ethylene oxide with alcohols, fatty acid esters, monoglyceride or diglycerides of long chain alcohols, and mixtures thereof. In one particular embodiment, the nonionic surfactant may be a fatty acid ester, such as a sucrose fatty acid ester, glycerol fatty acid ester, propylene glycol fatty acid ester, sorbitan fatty acid ester, pentaerythritol fatty acid ester, sorbitol fatty acid ester, and so forth. The fatty acid used to form such esters may be saturated or unsaturated, substituted or unsubstituted, and may contain from 6 to 22 carbon atoms, in some embodiments from 8 to 18 carbon atoms, and in some embodiments, from 12 to 14 carbon atoms. In one particular embodiment, mono- and di-glycerides of fatty acids may be employed in the present invention.

The thermoplastic starch may be formed using any of a variety of known techniques. For example, in one embodiment, the thermoplastic starch is formed prior to being combined with the biodegradable polyester, polyolefins, compatibilizers, colorants, etc. In such embodiments, the starch may be initially blended with the plasticizer, emulsifying surfactant, etc., to form the thermoplastic starch. Batch and/or continuous melt blending techniques may be employed. For example, a mixer/kneader, Banbury mixer, Farrel continuous mixer, single-screw extruder, twin-screw extruder, roll mill, etc., may be utilized to blend the materials. One particularly suitable melt-blending device is a co-rotating, twin-screw extruder (e.g., USALAB twin-screw extruder available from Thermo Electron Corporation of Stone, England or an extruder available from Werner-Pfleiderer from Ramsey, N.J.). Such extruders may include feeding and venting ports and provide high intensity distributive and dispersive mixing. For example, a starch composition may be initially fed to a feeding port of the twin-screw extruder. Thereafter, the plasticizer may be injected into the starch composition. Alternatively, the starch composition may be simultaneously fed to the feed throat of the extruder or separately at a different point along its length. Melt blending may occur at any of a variety of temperatures, such as from about 30° C. to about 200° C., in some embodiments, from about 40° C. to about 160° C., and in some embodiments, from about 50° C. to about 150° C.

### 3. Polyolefins

Examples of the polyolefins that may be incorporated in the present invention can include low-density polyethylene, high-density polyethylene, linear low-density polyethylene, polyolefin elastomers such as Vistamaxx from Exxon Mobil, or ethylene copolymers with vinyl acetate, or methacrylate, etc. A mixture of two or more polyolefins are also useful for this invention, as the combined polyolefins will provide a balanced profile of mechanical and physical properties.

The compatibilizer may include: ethylene vinyl acetate (EVA), ethylene vinyl alcohol (EVOH), polymer ethylene-co-acrylic acid, and a graft copolymer of non-polar polymer grafted with a polar monomer such as a polyethylene grafted with maleic anhydride. The polar functional monomer is maleic anhydride, acrylic acid, 2-hydroxyethyl methacrylate, glycidyl (meth)acrylate, vinyl acetate, vinyl alcohol, amino, amide, or acrylate. The polar functional monomer may be present in an amount that ranges from about 0.1% or 0.3% to about 40% or 45% by weight; desirably, about 0.5 wt. % or 1 wt. % to about 35 wt. % or 37 wt. %, inclusive. The composition may also contain from about 0.5% to about 30% of a biodegradable polymer.

The polymeric film can include a mineral filler that is present in an amount from about 5% or 8% to about 33% or 35% by weight, inclusive. Typically, the mineral filler is present in an amount from about 10% or 12% to about 25% or 30% by weight. The mineral filler may be selected from any one or a combination of the following: talcum powder, calcium carbonate, magnesium carbonate, clay, silica, alumina, boron oxide, titanium oxide, cerium oxide, germanium oxide, etc. The filler-containing film can be stretched to form breathable films.

The polymeric films and packaging can have multiple layers, for instance, from 1 to 7 or 8 layers; or in some embodiments, between about 2 or 3 to about 10 layers. The combined polymeric film layers can have a thickness of ranging from about 0.5 mil to about 5 mil, typically from about 0.7 or 1 mil to about 3 or 4 mil. Each layer can have a different composition, but at least one of the layers is formed from the present film composition. The at least one layer is formed with a thermoplastic starch concentrate such as a blend of thermoplastic starch, polyethylene and a compatibilizer with the high thermoplastic starch content, in some cases the TPS content can range from 50 to 90% by weight. The polyethylene in the layer can be low density polyethylene, linear low density polyethylene, high density polyethylene or ethylene copolymers, or mixtures of polyolefins. At least one layer on the seal side is polyethylene layer. Alternatively, a polymeric flexible film layer has a thickness from about 10 or 15 micrometers to about 90 or 100 micrometers. Typically, the film has a thickness from about 15 or 20 micrometer to about 45 or 50 micrometers. Desirably, the film thickness is about 15 to about 35 micrometers.

Generally, the flexible polymeric film according to the invention exhibits a modulus from about 50 MPa to about 300 Mpa, and a peak stress ranges from about 15 MPa to about 50 MPa, at an elongation of from about 200% to about 1000% of original dimensions. Typically, the modulus is in a range from about 55 or 60 MPa to about 260 or 275 MPa, and more typically from about 67 or 75 MPa to about 225 or 240 MPa, inclusive of any combination of ranges there between. Typically, the peak stress can range from about 20 or 23 MPa to about 40 or 45 MPa, inclusive of any combination of ranges there between.

The polymeric film will tend to have a micro-textured surface with topographic features, such as ridges or bumps, of between about 0.5 or 1 micrometers up to about 10 or 12 micrometers in size. Typically the features will have a dimension of about 2 or 3 micrometers to about 7 or 8 micrometers, or on average about 4, 5, or 6 micrometers. The particular size of the topographic features will tend to depend on the size of the individual starch particles, and/or their agglomerations.

### 4. Compatibilizers and Other Components

Other materials such as aliphatic polyesters can also be incorporated, as described in U.S. Patent Application Publication No. 2009-0203281A1, the content of which is incorporated herein by reference.

Further as described in Chinese Patent Application No. 2009-10146604.6, the content of which is incorporated herein by reference, compatibilizers can also be employed with the present film composition. To improve the compatibility and dispersion characteristics of TPS in polyolefins and biodegradable polyesters, several compatibilizers with both polar and non-polar groups are incorporated in the present invention. The compatibilizers may include several different kinds of copolymers, for example, polyethylene-co-vinyl acetate (EVA), polyethylene-co-vinyl alcohol (EVOH), polyethylene-co-acrylic (EAA), and a graft copolymer of a polyolefin (e.g., polyethylene) (e.g., DuPont Fusabond® MB-528D) and a polar monomer such as maleic anhydride, 2-hydroxyethyl methacrylate, acrylic acid, glycidyl (meth)acrylate, etc. EVA, EVOH, EAA, etc. both have a non-polar polyethylene subunit in their backbone. The vinyl acetate subunit contains an ester group, which associated with the hydroxyls of the amylopectin and amylose. Instead of the ester group from the vinyl acetate, EVOH has a vinyl alcohol group which has hydroxyl group as in starch. Both the ester group in EVA and the hydroxyl group in EVOH do not chemically react with the hydroxyl groups starch molecules. They only associate with starch through hydrogen bonding or polar-polar molecular interactions. Using these two physical compatibilizers, TPS and EVA or EVOH blends showed improved compatibility versus the un-compatibilized PE/TPS blends.

As a graft copolymer of polyethylene and maleic anhydride, Fusabond® MB-528D has a structure shown in FIG. 4:

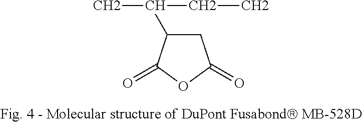

The cyclic anhydride at one end is chemically bonded directly into the polyethylene chain. The polar anhydride group of the molecule could associate with the hydroxyl groups in the starch via both hydrogen bonding and polar-polar molecular interactions and a chemical reaction to form an ester linkage during the melt extrusion process. The hydroxyls of the starch will undergo esterification reaction with the anhydride to achieve a ring-opening reaction to chemically link the TPS to the maleic anhydride to the grafted polyethylene. This reaction is accomplished under the high temperatures and pressures of the extrusion process.

For example, the DuPont Fusabond® MB-528D, at a concentration of about 1-5% completely dispersed the thermoplastic starch in the film. The EVA and EVOH worked sufficiently well to disperse the starch particles. In comparison to the graft copolymer of polyethylene and maleic anhydride, however, EVA and EVOH, even at higher percentages of around 10 or 15%, did not fully disperse the TPS in the film. Hence, the graft copolymer of polyethylene and maleic anhydride appears to be a more effective compatibilizer.

### B. Film Construction

The film of the present invention may be mono- or multi-layered. Multilayer films may be prepared by co-extrusion of the layers, extrusion coating, or by any conventional layering process. Such multilayer films normally contain at least one base layer and at least one skin layer, but may contain any number of layers desired. For example, the multilayer film may be formed from a base layer and one or more skin layers, wherein the base layer is formed from a blend of the biodegradable polyester and thermoplastic starch. In most embodiments, the skin layer(s) are formed from a biodegradable polyester and/or thermoplastic starch, such as described above. It should be understood, however, that other polymers may also be employed in the skin layer(s), such as polyolefin polymers (e.g., linear low-density polyethylene (LLDPE) or polypropylene). The term “linear low density polyethylene” refers to polymers of ethylene and higher alpha olefin comonomers, such as C₃-C₁₂ and combinations thereof, having a Melt Index (as measured by ASTM D-1238) of from about 0.5 to about 30 grams per 10 minutes at 190° C. Examples of predominately linear polyolefin polymers include, without limitation, polymers produced from the following monomers: ethylene, propylene, 1-butene, 4-methyl-pentene, 1-hexene, 1-octene and higher olefins as well as copolymers and terpolymers of the foregoing. In addition, copolymers of ethylene and other olefins including butene, 4-methyl-pentene, hexene, heptene, octene, decene, etc., are also examples of predominately linear polyolefin polymers. Additional film-forming polymers that may be suitable for use with the present invention, alone or in combination with other polymers, include ethylene vinyl acetate, ethylene ethyl acrylate, ethylene acrylic acid, ethylene methyl acrylate, ethylene normal butyl acrylate, nylon, ethylene vinyl alcohol, polystyrene, polyurethane, and so forth.

Any known technique may be used to form a film from the compounded material, including blowing, casting, flat die extruding, etc. In one particular embodiment, the film may be formed by a blown process in which a gas (e.g., air) is used to expand a bubble of the extruded polymer blend through an annular die. The bubble is then collapsed and collected in flat film form. Processes for producing blown films are described, for instance, in U.S. Pat. No. 3,354,506 to Raley; U.S. Pat. No. 3,650,649 to Schippers; and U.S. Pat. No. 3,801,429 to Schrenk et al., as well as U.S. Patent Application Publication Nos. 2005/0245162 to McCormack, et al. and 2003/0068951 to Boggs. et al., all of which are incorporated herein in their entirety by reference thereto for all purposes. In yet another embodiment, however, the film is formed using a casting technique.

Generally, the method of forming a film can involve: providing a polymer blend including a plasticized natural polymer, a biodegradable polymer, a polyolefin, and a compatibilizer with both a polar and a non-polar moiety on the same polymer molecule, where total biodegradable components in said cast film constitute at a majority phase of least 53 wt. % of dry polymer blend; mixing said polymer blend under melt extrusion conditions; extruding said polymer blend, and forming a film sheet.

For instance, according to an embodiment of a method for forming a cast film, the raw materials (e.g., biodegradable polyester, thermoplastic starch, etc.) may be supplied to a melt blending device, either separately or as a blend. In one embodiment, for example, a pre-formed thermoplastic starch and biodegradable polyester are separately supplied to a melt blending device where they are dispersively blended in a manner such as described above. For example, an extruder may be employed that includes feeding and venting ports. In one embodiment, the biodegradable polyester may be fed to a feeding port of the twin-screw extruder and melted. Thereafter, the thermoplastic starch may be fed into the polymer melt. Regardless, the materials are blended under high shear/pressure and heat to ensure sufficient mixing. For example, melt blending may occur at a temperature of from about 50° C. to about 300° C., in some embodiments, from about 70° C. to about 250° C., and in some embodiments, from about 90° C. to about 180° C. Likewise, the apparent shear rate during melt blending may range from about 100 seconds to about 10,000 seconds⁻¹, in some embodiments from about 500 seconds to about 5000 seconds⁻¹, and in some embodiments, from about 800 seconds⁻¹ to about 1200 seconds⁻¹. The apparent shear rate is equal to 4Q/πR³, where Q is the volumetric flow rate (“m³/s”) of the polymer melt and R is the radius (“m”) of the capillary (e.g., extruder die) through which the melted polymer flows.

Thereafter, the extruded material may be immediately chilled and cut into pellet form. In the particular, the compounded material can be then supplied to an extrusion apparatus and cast onto a casting roll to form a single-layered precursor film. If a multilayered film is to be produced, the multiple layers are co-extruded together onto the casting roll. The casting roll may optionally be provided with embossing elements to impart a pattern to the film. Typically, the casting roll is kept at temperature sufficient to solidify and quench the sheet as it is formed, such as from about 20 to 60° C. If desired, a vacuum box may be positioned adjacent to the casting roll to help keep the precursor film close to the surface of the roll. Additionally, air knives or electrostatic pinners may help force the precursor film against the surface of the casting roll as it moves around a spinning roll. An air knife is a device known in the art that focuses a stream of air at a very high flow rate to pin the edges of the film.

Once cast, the film may then be optionally oriented in one or more directions to further improve film uniformity and reduce thickness. Orientation may also form micropores in a film containing a filler, thus providing breathability to the film. For example, the film may be immediately reheated to a temperature below the melting point of one or more polymers in the film, but high enough to enable the composition to be drawn or stretched. In the case of sequential orientation, the “softened” film is drawn by rolls rotating at different speeds of rotation such that the sheet is stretched to the desired draw ratio in the longitudinal direction (machine direction). This “uniaxially” oriented film may then be laminated to a fibrous web. In addition, the uniaxially oriented film may also be oriented in the cross-machine direction to form a “biaxially oriented” film. For example, the film may be clamped at its lateral edges by chain clips and conveyed into a tenter oven. In the tenter oven, the film may be reheated and drawn in the cross-machine direction to the desired draw ratio by chain clips diverged in their forward travel.

For example, one method for forming a uniaxially oriented film is shown. As illustrated, the precursor film is directed to a film-orientation unit or machine direction orienter (“MDO”), such as commercially available from Marshall and Williams, Co. of Providence, R.I. The MDO has a plurality of stretching rolls (such as from 5 to 8) which progressively stretch and thin the film in the machine direction, which is the direction of travel of the film through the process. The MDO process can be performed with a number of rolls depending on the level of stretch that is desired and the degrees of stretching between each roll. The film may be stretched in either single or multiple discrete stretching operations. It should be noted that some of the rolls in an MDO apparatus may not be operating at progressively higher speeds. If desired, some of the rolls of the MDO may act as preheat rolls. If present, these first few rolls heat the film above room temperature (e.g., to 125° F.). The progressively faster speeds of adjacent rolls in the MDO act to stretch the film. The rate at which the stretch rolls rotate determines the amount of stretch in the film and final film weight.

The resulting film may then be wound and stored on a take-up roll. Various additional potential processing and/or finishing steps known in the art, such as slitting, treating, aperturing, printing graphics, or lamination of the film with other layers (e.g., nonwoven web materials), may be performed without departing from the spirit and scope of the invention.

The thickness of the resulting thin film may generally vary depending upon the desired use. Nevertheless, the film thickness is typically minimized to reduce the time needed for the film to disperse in water. Thus, in most embodiments of the present invention, the water-sensitive biodegradable film has a thickness of about 50 micrometers or less, in some embodiments from about 1 to about 40 micrometers, in some embodiments from about 2 to about 35 micrometers, and in some embodiments, from about 5 to about 30 micrometers.

Despite having such a small thickness and good sensitivity in water, the film of the present invention is nevertheless able to retain good dry mechanical properties during use. One parameter that is indicative of the relative dry strength of the film is the ultimate tensile strength, which is equal to the peak stress obtained in a stress-strain curve. Desirably, the film of the present invention exhibits an ultimate tensile strength in the machine direction (“MD”) of from about 10 to about 80 Megapascals (MPa), in some embodiments from about 15 to about 60 MPa, and in some embodiments, from about 20 to about 50 MPa, and an ultimate tensile strength in the cross-machine direction (“CD”) of from about 2 to about 40 Megapascals (MPa), in some embodiments from about 4 to about 40 MPa, and in some embodiments, from about 5 to about 30 MPa. Although possessing good strength, it is also desirable that the film is not too stiff. One parameter that is indicative of the relative stiffness of the film is Young's modulus of elasticity, which is equal to the ratio of the tensile stress to the tensile strain and is determined from the slope of a stress-strain curve. For example, the film typically exhibits a Young's modulus in the machine direction (“MD”) of from about 50 to about 1200 Megapascals (“MPa”), in some embodiments from about 200 to about 1000 MPa, and in some embodiments, from about 400 to about 800 MPa, and a Young's modulus in the cross-machine direction (“CD”) of from about 50 to about 1000 Megapascals (“MPa”), in some embodiments from about 100 to about 800 MPa, and in some embodiments, from about 150 to about 500 MPa.

The film of the present invention may be used in a wide variety of applications. For example, as indicated above, the film may be used in an absorbent article. An “absorbent article” generally refers to any article capable of absorbing water or other fluids. Examples of some absorbent articles include, but are not limited to, personal care absorbent articles, such as diapers, training pants, absorbent underpants, incontinence articles, feminine hygiene products (e.g., sanitary napkins, pantiliners, etc.), swim wear, baby wipes, and so forth; medical absorbent articles, such as garments, fenestration materials, underpads, bedpads, bandages, absorbent drapes, and medical wipes; food service wipers; clothing articles; and so forth. Several examples of such absorbent articles are described in U.S. Pat. No. 5,649,916 to DiPalma, et al.; U.S. Pat. No. 6,110,158 to Kielpikowski; U.S. Pat. No. 6,663,611 to Blaney, et al., which are incorporated herein in their entirety by reference thereto for all purposes. Still other suitable articles are described in U.S. Patent Application Publication No. 2004/0060112 A1 to Fell et al., as well as U.S. Pat. No. 4,886,512 to Damico et al.; U.S. Pat. No. 5,558,659 to Sherrod et al.; U.S. Pat. No. 6,888,044 to Fell et al.; and U.S. Pat. No. 6,511,465 to Freiburger et al., all of which are incorporated herein in their entirety by reference thereto for all purposes. Materials and processes suitable for forming such absorbent articles are well known to those skilled in the art.

Absorbent article may be provided with adhesives (e.g., pressure-sensitive adhesives) that help removably secure the article to the crotch portion of an undergarment and/or wrap up the article for disposal. Suitable pressure-sensitive adhesives, for instance, may include acrylic adhesives, natural rubber adhesives, tackified block copolymer adhesives, polyvinyl acetate adhesives, ethylene vinyl acetate adhesives, silicone adhesives, polyurethane adhesives, thermosettable pressure-sensitive adhesives, such as epoxy acrylate or epoxy polyester pressure-sensitive adhesives, etc. Such pressure-sensitive adhesives are known in the art and are described in the Handbook of Pressure Sensitive Adhesive Technology, Satas (Donatas), 1989, 2^{nd} edition, Van Nostrand Reinhold. The pressure sensitive adhesives may also include additives such as cross-linking agents, fillers, gases, blowing agents, glass or polymeric microspheres, silica, calcium carbonate fibers, surfactants, and so forth. The additives are included in amounts sufficient to affect the desired properties.

The location of the adhesive on the absorbent article is not critical and may vary widely depending on the intended use of the article. For example, certain feminine hygiene products (e.g., sanitary napkins) may have wings or flaps that laterally from a central absorbent core and are intended to be folded around the edges of the wearer's panties in the crotch region. The flaps may be provided with an adhesive (e.g., pressure-sensitive adhesive) for affixing the flaps to the underside of the wearer's panties. Regardless of the particular location of the adhesive, however, a release liner may be employed to cover the adhesive, thereby protecting it from dirt, drying out, and premature sticking prior to use. The release liner may contain a release coating that enhances the ability of the liner to be peeled from an adhesive.

The release coating contains a release agent, such as a hydrophobic polymer. Exemplary hydrophobic polymers include, for instance, silicones (e.g., polysiloxanes, epoxy silicones, etc.), perfluoroethers, fluorocarbons, polyurethanes, and so forth. Examples of such release agents are described, for instance, in U.S. Pat. No. 6,530,910 to Pomplun, et al.; U.S. Pat. No. 5,985,396 to Kerins, et al.; and U.S. Pat. No. 5,981,012 to Pomplun, et al., which are incorporated herein in their entirety by reference thereto for all purposes. One particularly suitable release agent is an amorphous polyolefin having a melt viscosity of about 400 to about 10,000 cps at 190° C., such as made by the U.S. Rexene Company under the tradename REXTAC® (e.g., RT2315, RT2535 and RT2330). The release coating may also contain a detackifier, such as a low molecular weight, highly branched polyolefin. A particularly suitable low molecular weight, highly branched polyolefin is VYBAR® 253, which is made by the Petrolite Corporation. Other additives may also be employed in the release coating, such as compatibilizers, processing aids, plasticizers, tackifiers, slip agents, and antimicrobial agents, and so forth. The release coating may be applied to one or both surfaces of the liner, and may cover all or only a portion of a surface. Any suitable technique may be employed to apply the release coating, such as solvent-based coating, hot melt coating, solventless coating, etc. Solvent-based coatings are typically applied to the release liner by processes such as roll coating, knife coating, curtain coating, gravure coating, wound rod coating, and so forth. The solvent (e.g., water) is then removed by drying in an oven, and the coating is optionally cured in the oven. Solventless coatings may include solid compositions, such as silicones or epoxy silicones, which are coated onto the liner and then cured by exposure to ultraviolet light. Optional steps include priming the liner before coating or surface modification of the liner, such as with corona treatment. Hot melt coatings, such as polyethylenes or perfluoroethers, may be heated and then applied through a die or with a heated knife. Hot melt coatings may be applied by co-extruding the release agent with the release liner in blown film or sheet extruder for ease of coating and for process efficiency.

To facilitate its ability to be easily disposed, the release liner may be formed from a film in accordance with the present invention. In this regard, one particular embodiment of a sanitary napkin that may employ the film of the present invention will now be described in more detail. For purposes of illustration only, an absorbent article can be a sanitary napkin for feminine hygiene. In such an embodiment, the absorbent article includes a main body portion containing a topsheet, an outer cover or backsheet, an absorbent core positioned between the backsheet and the topsheet, and a pair of flaps extending from each longitudinal side of the main body portion. The topsheet defines a bodyfacing surface of the absorbent article. The absorbent core is positioned inward from the outer periphery of the absorbent article and includes a body-facing side positioned adjacent the topsheet and a garment-facing surface positioned adjacent the backsheet.

The topsheet is generally designed to contact the body of the user and is liquid-permeable. The topsheet may surround the absorbent core so that it completely encases the absorbent article. Alternatively, the topsheet and the backsheet may extend beyond the absorbent core and be peripherally joined together, either entirely or partially, using known techniques. Typically, the topsheet and the backsheet are joined by adhesive bonding, ultrasonic bonding, or any other suitable joining method known in the art. The topsheet is sanitary, clean in appearance, and somewhat opaque to hide bodily discharges collected in and absorbed by the absorbent core. The topsheet further exhibits good strike-through and rewet characteristics permitting bodily discharges to rapidly penetrate through the topsheet to the absorbent core, but not allow the body fluid to flow back through the topsheet to the skin of the wearer. For example, some suitable materials that may be used for the topsheet include nonwoven materials, perforated thermoplastic films, or combinations thereof. A nonwoven fabric made from polyester, polyethylene, polypropylene, bicomponent, nylon, rayon, or like fibers may be utilized. For instance, a white uniform spunbond material is particularly desirable because the color exhibits good masking properties to hide menses that has passed through it. U.S. Pat. No. 4,801,494 to Datta, et al. and U.S. Pat. No. 4,908,026 to Sukiennik, et al., teach various other cover materials that may be used in the present invention.

The topsheet may also contain a plurality of apertures (not shown) formed therethrough to permit body fluid to pass more readily into the absorbent core. The apertures may be randomly or uniformly arranged throughout the topsheet, or they may be located only in the narrow longitudinal band or strip arranged along the longitudinal axis of the absorbent article. The apertures permit rapid penetration of body fluid down into the absorbent core. The size, shape, diameter and number of apertures may be varied to suit one's particular needs.

As stated above, the absorbent article also includes a backsheet. The backsheet is generally liquid-impermeable and designed to face the inner surface, i.e., the crotch portion of an undergarment (not shown). The backsheet may permit a passage of air or vapor out of the absorbent article, while still blocking the passage of liquids. Any liquid-impermeable material may generally be utilized to form the backsheet. For example, one suitable material that may be utilized is a microembossed polymeric film, such as polyethylene or polypropylene. In particular embodiments, a polyethylene film is utilized that has a thickness in the range of about. 0.2 mils to about 5.0 mils, and particularly between about 0.5 to about 3.0 mils.

The absorbent article also contains an absorbent core positioned between the topsheet and the backsheet. The absorbent core may be formed from a single absorbent member or a composite containing separate and distinct absorbent members. It should be understood, however, that any number of absorbent members may be utilized in the present invention. For example, in an embodiment, the absorbent core may contain an intake member (not shown) positioned between the topsheet and a transfer delay member (not shown). The intake member may be made of a material that is capable of rapidly transferring, in the z-direction, body fluid that is delivered to the topsheet. The intake member may generally have any shape and/or size desired. In one embodiment, the intake member has a rectangular shape, with a length equal to or less than the overall length of the absorbent article, and a width less than the width of the absorbent article. For example, a length of between about 150 mm to about 300 mm and a width of between about 10 mm to about 60 mm may be utilized.

Any of a variety of different materials may be used for the intake member to accomplish the above-mentioned functions. The material may be synthetic, cellulosic, or a combination of synthetic and cellulosic materials. For example, airlaid cellulosic tissues may be suitable for use in the intake member. The airlaid cellulosic tissue may have a basis weight ranging from about 10 grams per square meter (gsm) to about 300 gsm, and in some embodiments, between about 100 gsm to about 250 gsm. In one embodiment, the airlaid cellulosic tissue has a basis weight of about 200 gsm. The airlaid tissue may be formed from hardwood and/or softwood fibers. The airlaid tissue has a fine pore structure and provides an excellent wicking capacity, especially for menses.

If desired, a transfer delay member (not shown) may be positioned vertically below the intake member. The transfer delay member may contain a material that is less hydrophilic than the other absorbent members, and may generally be characterized as being substantially hydrophobic. For example, the transfer delay member may be a nonwoven fibrous web composed of a relatively hydrophobic material, such as polypropylene, polyethylene, polyester or the like, and also may be composed of a blend of such materials. One example of a material suitable for the transfer delay member is a spunbond web composed of polypropylene, multi-lobal fibers. Further examples of suitable transfer delay member materials include spunbond webs composed of polypropylene fibers, which may be round, tri-lobal or poly-lobal in cross-sectional shape and which may be hollow or solid in structure. Typically the webs are bonded, such as by thermal bonding, over about 3% to about 30% of the web area. Other examples of suitable materials that may be used for the transfer delay member are described in U.S. Pat. No. 4,798,603 to Meyer, et al. and U.S. Pat. No. 5,248,309 to Serbiak, et al., which are incorporated herein in their entirety by reference thereto for all purposes. To adjust the performance of the invention, the transfer delay member may also be treated with a selected amount of surfactant to increase its initial wettability.

The transfer delay member may generally have any size, such as a length of about 150 mm to about 300 mm. Typically, the length of the transfer delay member is approximately equal to the length of the absorbent article. The transfer delay member may also be equal in width to the intake member, but is typically wider. For example, the width of the transfer delay member may be from between about 50 mm to about 75 mm, and particularly about 48 mm. The transfer delay member typically has a basis weight less than that of the other absorbent members. For example, the basis weight of the transfer delay member is typically less than about 150 grams per square meter (gsm), and in some embodiments, between about 10 gsm to about 100 gsm. In one particular embodiment, the transfer delay member is formed from a spunbonded web having a basis weight of about 30 gsm.

Besides the above-mentioned members, the absorbent core may also include a composite absorbent member (not shown), such as a coform material. In this instance, fluids may be wicked from the transfer delay member into the composite absorbent member. The composite absorbent member may be formed separately from the intake member and/or transfer delay member, or may be formed simultaneously therewith. In one embodiment, for example, the composite absorbent member may be formed on the transfer delay member or intake member, which acts a carrier during the coform process described above.

Regardless of its particular construction, the absorbent article typically contains an adhesive for securing to an undergarment. An adhesive may be provided at any location of the absorbent article, such as on the lower surface of the backsheet. In this particular embodiment, the backsheet carries a longitudinally central strip of garment adhesive covered before use by a peelable release liner, which may be formed in accordance with the present invention. Each of the flaps may also contain an adhesive positioned adjacent to the distal edge of the flap. A peelable release liner, which may also be formed in accordance with the present invention, may cover the adhesive before use. Thus, when a user of the sanitary absorbent article wishes to expose the adhesives and secure the absorbent article to the underside of an undergarment, the user simply peels away the liners and disposed them in a water-based disposal system (e.g., in a toilet).

Although various configurations of a release liner have been described above, it should be understood that other release liner configurations are also included within the scope of the present invention. Further, the present invention is by no means limited to release liners and the water-sensitive biodegradable film may be incorporated into a variety of different components of an absorbent article. For example, the backsheet of the napkin may include the water-sensitive film of the present invention. In such embodiments, the film may be used alone to form the backsheet or laminated to one or more additional materials, such as a nonwoven web. The water-sensitive biodegradable film of the present invention may also be used in applications other than absorbent articles. For example, the film may be employed as an individual wrap, packaging pouch, or bag for the disposal of a variety of articles, such as food products, absorbent articles, etc. Various suitable pouch, wrap, or bag configurations for absorbent articles are disclosed, for instance, in U.S. Pat. No. 6,716,203 to Sorebo. et al. and U.S. Pat. No. 6,380,445 to Moder, et al., as well as U.S. Patent Application Publication No. 2003/0116462 to Sorebo, et al., all of which are incorporated herein in their entirety by reference thereto for all purposes.

### Section III

### EXAMPLES

The following section details some comparative film samples that better illustrate and distinguish the examples of films made according to the present invention. Through extensive experimental investigation, the compositions in the working range were defined in the following examples. However, just the compositions alone are not enough to produce a formulation with the right performance characteristics and processability required for making thin film layers. The method of processing is also important to achieve high performance and desired processability.

### Comparative Example 1

A thermoplastic starch (TPS) was made from native corn starch (NCS) at with 25% by weight of glycerin in on the ZSK-30 extruder (Werner and Pfleiderer Corporation, Ramsey, N.J.) which is a co-rotating, twin screw extruder, with a diameter of 30 mm and screw length of 1328 mm. The extruder has 14 barrels. The extruder was coupled into 7 heating zones. The temperatures of the heating zones were respectively 70, 80, 140, 150, 150, 150, and 150° C. Before compounding, Excel P40-S was added (2% by weight) to the native corn starch (NCS), the mixed starch was fed to the feed throat of the extruder which was not heated at a rate of 12 lbs/hr using a gravimetric feeder. The glycerin was warmed in order to achieve a pump delivery rate needed for the desired level of glycerin. Glycerin was injected into barrel 2 with a pressurized injector at a rate of 4 lbs/hr using an Eldex pump (Napa, Calif.). The screw speed was 160 rpm, the melt temperature was measured to range from 125 to 130° C. The melt process was from 420 to 800 psi during the extrusion. The torque ranged from 27 to 43%. The process conditions were summarized in Table 1 as well. The converted thermoplastic starch stands were cooled on a fan-cooled conveyor belt and then pelletized. The pelletized TPS was then used to make resins for future film casting.

TABLE 1
Compositions of Resins of all Examples

| | | | | Ampacet |
|---|---|---|---|---|
| | | | | TiO₂ |
| | | Component | | masterbatch |
| Sample ID | Resin Components | Ratios (%) | TiO₂ (%) | (%) |
| Comparative | Native corn starch:glycerin | 75:25 | 0 | 0 |
| Example 1 | | | | |
| | Ecoflex:TPS* | 60:40 | 0 | 0 |
| Comparative | Ecoflex:PE | 65:35 | 0 | 0 |
| Example 3 | | | | |
| Comparative | Ecoflex:PE | 65:35 | 2 | 0 |
| Example 4 | | | | |
| Example 1 | ETPS:Dowlex:Fusabond | 63.375:34.125:2.5 | 0 | 0 |
| Example 2 | ETPS:EVA:Fusabond | 63.375:34.125:2.5 | 0 | 0 |
| Example 3 | ETPS:PE | 65:35 | 2 | 0 |
| Example 4 | ETPS:EVA | 65:35 | 2 | 0 |
| Example 5 | ETPS:PE | 60:40 | 2 | 0 |
| Example 6 | ETPS:EVA | 60:40 | 2 | 0 |
| Example 7 | Ecoflex:TPS | 60:40 | 2 | 0 |
| Example 8 | ETPS:PE:Fusabond | 63.375:34.125:2.5 | 2 | 0 |
| Example 9 | ETPS:EVA:Fusabond | 63.375:34.125:2.5 | 2 | 0 |
| Example 10 | ETPS:PE:Fusabond | 63.375:34.125:2.5 | 0 | 5 |
| Example 11 | ETPS:PE | 65:35 | 0 | 5 |

*The Ecoflex:TPS (60:40), referred to as ETPS, was used to make samples in other examples.

Cargill Gel Corn Starch was purchased from Cargill (Cedar Rapids, Iowa).
Glycerin, a processing aid, was purchased from Cognis Corporation (Cincinnati, Ohio).
Excel P-40S, a hydrogenated glyceride used as a surfactant for resin compounding, was purchased from Kao Corporation (Tokyo, Japan).
Ecoflex™ F BX 7011, an aliphatic aromatic copolyester, was purchased from BASF (Ludwigshafen, Germany), designated as Ecoflex in the table for short.
Dowlex EG 2244G polyethylene resin was purchased from Dow Chemical Company (Midland, Mich.), designated as PE.
Escorene Ultra LD 755.12, and an ethylene vinyl acetate (EVA) copolymer, was purchased from ExxonMobil Chemical Company (Houston, Tex.).
Fusabone MB 528D, a chemically modified polyethylene resin, was purchased from DuPont Company (Wilmington, Del.), designated as FB.
Dupont Ti-Pure titanium dioxide was purchased from DuPont Company (Wilmington, Del.), designated as TiO₂.
Ampacet 110313 B White PE, a white colorant, was purchased from Ampacet Corporation (Terre Haute, Ind.), designated as Amp.

### Comparative Example 2

The same equipment set as in Comparative Example 1 was used for making this sample. In this example, Ecoflex F BX 7011 from BASF fed at a rate of 15 lbs/hr via a gravimetric feeder, and TPS made from Comparative Example 1 was fed at a rate of 10 lbs/hr, respectively to the feed-throat of the extruder. The conditions for preparing this example are listed in Table 1. The melt temperature was observed to be from 148 to 155° C. The resulting blend was designated as ETPS.

### Comparative Example 3

This example was made from Ecoflex:PE in a 65:35 ratio. Ecoflex and PE were placed in separate feeders and fed into barrel 1 of the extruder. Ecoflex was fed at a rate of 13 lb/h, and the PE was fed at a rate of 7 lb/h. The resulting ETPS extrudate strands were cooled on a moving belt and pelletized in order to cast films and to blend with other resins.

### Comparative Example 4

Ecoflex, PE, and titanium oxide were placed in separate feeders and fed into barrel 1 of the extruder. Ecoflex was fed at a rate of 13 lb/h, and the PE was fed at a rate of 7 lb/h, the 2% TiO2 was fed at 0.4 lb/hr (Code 10). The resulting extrudate strands were cooled on a moving belt and pelletized for use in film casting.

### Example 1

ETPS:PE:FB was made in approximately 63:34:3 ratios. The separate components for each blend were fed into barrel 1 of the extruder using separate feeders. ETPS was fed at a rate of 13 lb/h (unable to feed at the desired rate of 12.675 lb/h), the PE was fed at a rate of 6.825 lb/h, and the FB was fed at a rate of 0.5 lb/h. The detailed process conditions including screw speed, feed rate, set temperatures of the extruder, melt temperature, melt pressure, and torque were listed in Table 2. Surprisingly, the resulting strands had smooth surface and very strong indicating excellent preliminary compatibility. The resulting extrudate strands were cooled on a moving belt and pelletized in order to cast films.

### Example 2

ETPS:EVA:FB was made in approximately 63:34:3 ratios. The separate components for each blend were fed into barrel 1 of the extruder using separate feeders. ETPS was fed at a rate of 13 lb/h (unable to feed at the desired rate of 12.675 lb/h), the EVA was fed at a rate of 6.825 lb/h, and the FB was fed at a rate of 0.5 lb/h. The detailed process conditions including screw speed, feed rate, set temperatures of the extruder, melt temperature, melt pressure, and torque were listed in Table 2. Once again, smooth strands were surprisingly obtained. The strands were softer and more flexible than the strands obtained from Example 1. The resulting extrudate strands were cooled on a moving belt and pelletized in order to cast films.

### Example 3

ETPS:PE blend was made in 65:35 ratio containing 2% TiO₂ (Code 3). ETPS, PE, and TiO₂ were placed in separate feeders and fed into barrel 1 of the extruder. For the 65:35 blend, ETPS was fed at 13 lb/h, PE at 7 lb/h, and TiO₂ at 0.4 lb/h. The detailed process conditions including screw speed, feed rate, set temperatures of the extruder, melt temperature, melt pressure, and torque were listed in Table 2. The resulting extrudate strands were cooled on a moving belt and pelletized in order to cast films.

TABLE 2
Processing Conditions for Making Thermoplastic Starch Blends for Biodegradable Films on the ZSK-30

| | | Feed | | Screw | | | |
|---|---|---|---|---|---|---|---|
| | | Rate | Set Temperatures | Speed | Tmelt | Pmelt | Torque |
| Sample ID | Final Resin Composition | (lb/h) | (° C.) | (rpm) | (° C.) | (psi) | (%) |
| Comparative | NCS:Gly | 16* | 70, 80, 140, 150, | 160 | 130-125 | 420-800 | 27-43 |
| Example 1 | (75:25) | | 150, 150, 150 | | | | |
| Comparative | Ecoflex:NCS:Gly | 25 | 70, 80, 140, 145, | 150 | 148-155 | 260-320 | 58-63 |
| Example 2 | (60:30:10) | | 145, 145, 150 | | | | |
| Comparative | Ecoflex:PE | 20 | 70, 120, 150, 155, | 150 | 157-175 | 160-190 | 40-43 |
| Example 3 | (65:35) | | 160, 160, 160 | | | | |
| Example 1 | Ecoflex:NCS:Gly:PE:FB | 20 | 70, 80, 140, 150, | 150 | 155-164 | 250-290 | 39-43 |
| | (38:19:6:34:3)** | | 150, 150, 150 | | | | |
| Example 2 | Ecoflex:NCS:Gly:EVA:FB | 20 | 70, 80, 140, 150, | 150 | 152-163 | 150-200 | 38-42 |
| | (38:19:6:34:3)** | | 150, 150, 150 | | | | |
| Example 3 | Ecoflex:NCS:Gly:PE:TiO₂ | 20.4 | 70, 80, 140, 145, | 150 | 166-179 | 190-270 | 40-46 |
| | (~38:19:6.5:34.5:2) | | 145, 145. 150 | | | | |
| Example 4 | Ecoflex:NCS:Gly:EVA:TiO₂ | 20.4 | 70, 80, 140, 145, | 150 | 167-174 | 140-170 | 39-43 |
| | (~38:19:6.5:34.5:2) | | 145, 145. 150 | | | | |
| Example 5 | Ecoflex:NCS:Gly:PE:TiO₂ | 20.4 | 70, 80, 140, 145, | 150 | 165-179 | 210-220 | 42-50 |
| | (~35:18:6:39:2) | | 145, 145. 150 | | | | |
| Example 6 | Ecoflex:NCS:Gly:EVA:TiO₂ | 20.4 | 70, 80, 140, 145, | 150 | 167-179 | 140-170 | 39-42 |
| | (~35:18:6:39:2) | | 145, 145. 150 | | | | |
| Example 7 | Ecoflex:NCS:Gly:TiO₂ | 20.4 | 70, 80, 140, 145, | 150 | 165-179 | 180-230 | 50-56 |
| | (59:29:10:2) | | 145, 145. 150 | | | | |
| Example 8 | Ecoflex:NCS:Gly:PE:FB:TiO₂ | 20.725 | 70, 80, 140, 145, | 150 | 158-166 | 230-240 | 42-45 |
| | (~37:19:6:33:3:2) | | 145, 145. 150 | | | | |
| Example 9 | Ecoflex:NCS:Gly:EVA:FB:TiO₂ | 20.725 | 70, 80, 140, 145, | 150 | 165-168 | 150-230 | 42-44 |
| | (~37:19:6:33:3:2) | | 145, 145. 150 | | | | |
| Example 10 | Ecoflex:NCS:Gly:PE:FB:Amp | 21.325 | 70, 80, 140, 145, | 150 | 163-182 | 190-300 | 46-51 |
| | (~36:18:6:32:3:5) | | 145, 145, 150 | | | | |
| Example 11 | Ecoflex:NCS:Gly:PE:Amp | 21.0 | 70, 80, 140, 145, | 150 | 163-180 | 190-220 | 45-49 |
| | (~37:18:7:33:5) | | 145, 145, 150 | | | | |

*Glycerin (Gly) was pumped at a rate of 30.2 g/min (=4 lb/h).
**Actual ratios are 38.025/19.0125/6.3375/34.125/2.5.

### Example 4

ETPS:EVA blend were made in 65:35 containing 2% TiO₂. ETPS, EVA, and TiO₂ were placed in separate feeders and fed into barrel 1 of the extruder. For this blend, ETPS was fed at 13 lb/h, EVA at 7 lb/h, and TiO₂ at 0.4 lb/h. The detailed process conditions including screw speed, feed rate, set temperatures of the extruder, melt temperature, melt pressure, and torque were listed in Table 2. The resulting extrudate strands were cooled on a moving belt and pelletized in order to cast films.

### Example 5

ETPS:PE blend was made in 60:40 ratios containing 2% TiO₂. ETPS, PE, and TiO2 were placed in separate feeders and fed into barrel 1 of the extruder. For this blend, ETPS was fed at 12 lb/h, PE at 8 lb/h, and TiO₂ at 0.4 lb/h. The detailed process conditions including screw speed, feed rate, set temperatures of the extruder, melt temperature, melt pressure, and torque were listed in Table 2. The resulting extrudate strands were cooled on a moving belt and pelletized in order to cast films.

### Example 6

ETPS:EVA blend was made in 60:40 ratio containing 2% TiO₂. ETPS, EVA, and TiO₂ were placed in separate feeders and fed into barrel 1 of the extruder. For this 60:40 blend, ETPS was fed at 12 lb/h, EVA at 8 lb/h, and TiO₂ at 0.4 lb/h. The detailed process conditions including screw speed, feed rate, set temperatures of the extruder, melt temperature, melt pressure, and torque were listed in Table 2. The resulting extrudate strands were cooled on a moving belt and pelletized in order to cast films.

### Example 7

Ecoflex: TPS:TiO₂ (60:40:2) was prepared similar to Example 6. The detailed process conditions including screw speed, feed rate, set temperatures of the extruder, melt temperature, melt pressure, and torque were listed in Table 2. The resulting extrudate strands were cooled on a moving belt and pelletized in order to cast films.

### Example 8

The resin containing 2% TiO₂ was also made using the same ratios of ETPS:PE:FB. For Code 11, the Fusabond was dry blended with the Dowlex and EVA at a ratio of 6.8:93.2 and fed into barrel 1 of the extruder at a rate of 7.325 lb/h. The detailed process conditions including screw speed, feed rate, set temperatures of the extruder, melt temperature, melt pressure, and torque were listed in Table 2. The resulting extrudate strands were cooled on a moving belt and pelletized for use in film casting.

### Example 9

The resin containing 2% TiO₂ was also made using the same ratios of ETPS:EVA:FB. For this example, the Fusabond was dry blended with the Dowlex and EVA at a ratio of 6.8:93.2 and fed into barrel 1 of the extruder at a rate of 7.325 lb/h. The detailed process conditions including screw speed, feed rate, set temperatures of the extruder, melt temperature, melt pressure, and torque were listed in Table 2. The resulting extrudate strands were cooled on a moving belt and pelletized for use in film casting.

### Example 10

A resin containing Ampacet TiO₂ concentrate instead of TiO₂ was made. The composition was an approximate 63:34:3 ratio of ETPS/PE/Fusabond with 5% Ampacet added. The ETPS, PE/Fusabond (˜93/7), and Ampacet were placed into separate feeders and fed into barrel 1 of the extruder at rates of 13.0 lb/h, 7.325 lb/h, and 1.0 lb/h, respectively. The detailed process conditions including screw speed, feed rate, set temperatures of the extruder, melt temperature, melt pressure, and torque were listed in Table 2. The resulting extrudate strands were cooled on a moving belt and pelletized in order to cast films.

### Example 11

The resin containing Ampacet instead of TiO₂ were made. This example was a 65:35 blend of ETPS:PE with 5% Ampacet added. The ETPS, PE, and Ampacet were placed into separate feeders and fed into barrel 1 of the extruder at rates of 13.0 lb/h, 7.0 lb/h, and 1.0 lb/h, respectively. The detailed process conditions including screw speed, feed rate, set temperatures of the extruder, melt temperature, melt pressure, and torque were listed in Table 2. The resulting extrudate strands were cooled on a moving belt and pelletized in order to cast films.

### Film Casting

### Example 12

The resin blends made on the ZSK-30 extruder were used to cast films. Additional control films were also cast using 100% Ecoflex, Dowlex EG 2244G PE, and EVA 755.12 resins. Film casting was performed on a single screw extruder-HAAKE Rheomex 252 (Haake, Karlsruhe, Germany with a diameter of 18.75 mm and a screw length of 450 mm and an attached 4-inch film die. The extruder screws were driven by a Haake Rheocord 90. The ETPS:PE:FB, Ecoflex, Dowlex EG 2244G PE, and EVA 755.12 resins were flood (direct) fed into the extruder. ETPS, ETPS:EVA:FB, and Ecoflex:Dowlex EG 2244G PE resins were fed into the extruder using K-Tron pellet feeders (K-Tron Corporation, Pitman, N.J.). The resulting films were run through a Haake TP1 before being collected.

All films were successfully cast using conditions described in Table 3, which also lists final film compositions, melt temperature, and torque. The average thicknesses of the films ranged from approximately 0.6 mil to 1.50 mil.

TABLE 3
Conditions for Casting Biodegradable Films on the HAAKE Rheocord 90

| | | Set | Screw | | |
|---|---|---|---|---|---|
| | | Temperatures | Speed | Tmelt | Torque |
| Sample No. | Final Film Composition | (° C.) | (rpm) | (° C.) | (m.g) |
| Comprative | Ecoflex:NCS:Gly | 150, 165, | 50 | 163-166 | 432 |
| Example 2 | (60:30:10) | 165, 165, 155 | | | |
| Comparative | Ecoflex:PE | 155, 175, | 70 | 181-187 | 826-954 |
| Example 3 | (65:35) | 185, 185, 175 | | | |
| Comparative | Ecoflex (100) | 140, 150, | 20 | 154-161 | 900-1000 |
| Example 5 | | 160, 160, 150 | | | |
| Comparative | PE (100) | 155, 175, | 40 | 183-186 | 2199-2981 |
| Example 6 | | 185, 185, 175 | | | |
| Comparative | EVA (100) | 155, 175, | 40 | 180-188 | 1257-1278 |
| Example 7 | | 185, 185, 175 | | | |
| Example1 | Ecoflex:NCS:Gly:PE:FB | 155, 175, | 50-55 | 181-188 | 2819-2834 |
| | (38:19:6:34:3) | 185, 185, 175 | | | |
| Example 2 | Ecoflex:NCS:Gly:EVA:FB | 155, 175, | 50 | 183-187 | 389-479 |
| | (38:19:6:34:3) | 185, 185, 175 | | | |
| Example 3 | Ecoflex:NCS:Gly:PE:TiO₂ | 150, 155, | 80 | 162-165 | 1283-1639 |
| | (~38:19:6.5:34.5:2) | 160, 160, 160 | | | |
| Example 4 | Ecoflex:NCS:Gly:EVA:TiO₂ | 150, 155, | 160 | 161-168 | 1377-5823 |
| | (~38:19:6.5:34.5:2) | 160, 160, 160 | | | |
| Example 5 | Ecoflex:NCS:Gly:PE:TiO₂ | 150, 155, | 80 | 161-166 | 1304-2103 |
| | (~35:18:6:39:2) | 160, 160, 160 | | | |
| Example 6 | Ecoflex:NCS:Gly:EVA:TiO₂ | 150, 155, | 200 | 162-168 | 500-628 |
| | (~35:18:6:39:2) | 160, 160, 160 | | | |
| Example 7 | Ecoflex:NCS:Gly:TiO₂ | 150, 155, | 100 | 162-166 | 708-772 |
| | (59:29:10:2) | 160, 160, 160 | | | |
| Example 8 | Ecoflex:NCS:Gly:PE:FB:TiO₂ | 160, 165, | 80 | 176-178 | 724-841 |
| | (~37:19:6:33:3:2) | 170, 170, 170 | | | |
| Example 9 | Ecoflex:NCS:Gly:EVA:FB:TiO₂ | 160, 165, | 80 | 174-178 | 863-905 |
| | (~37:19:6:33:3:2) | 170, 170, 170 | | | |
| Example 10 | Ecoflex:NCS:Gly:PE:FB:Amp | 160, 165, | 85 | 171-191 | 1193-1353 |
| | (~36:18:6:32:3:5) | 170, 170, 175 | | | |
| Example 11 | Ecoflex:NCS:Gly:PE:Amp | 155, 160, | 80 | 166-187 | 959-1246 |
| | (~37:18:7:33:5) | 165, 165, 170 | | | |

The ETPS film (Comparative Example 2) was smooth film with a milky white coloring. The set temperatures were slightly increased to a maximum temperature of 165° C. (from an initial maximum temperature of 150° C.). The resin pellets were originally flood fed into the extruder, but this caused the extruder to bridge up, so the resin was then fed into the extruder using a pellet feeder.

Comparative Example 3 (Ecoflex:PE) film was a somewhat translucent, milky white, soft, stretchable film. The temperature of the extruder had to be increased to a maximum temperature of 185° C. (initial maximum temperature was 160° C.), due to the presence of unmelted particles in the film, which were causing holes. There was some surging of material occurring, causing the pressure at time to fluctuate. An ion air knife was placed over the initial set of rollers on the Haake T1 to help decrease the thickness of the film. Issues experienced while casting Control 2 were believed to be caused by incompatibility of the film components.

The Ecoflex (Comparative Example 5), Dowlex 2244G PE (Comparative Example 5), and the EVA 755.12 (Comparative Example 6) films were all flexible, clear and smooth films. Ecoflex and EVA films were very sticky, making it somewhat difficult to collect film samples, even with the use of release paper. The Dowlex film was slightly thicker on the edges and was a little sticky.

The film of Example 1 appeared to be smooth, flexible, and off-white in color. During initial film casting there were tiny, black particles present in the film, which eventually disappeared following further casting. It is not known if these particles were present in the actual resin or if they were burnt resin that had built up in the film die from previous experiments. Small holes were also located sporadically throughout the film. These holes were caused by the presence of unmelted resin particles in the film, which temperature adjustments to the extruder did not remedy.

The film of Example 2 was somewhat translucent, smooth, flexible, soft, and off-white in color. There were occasional issues with the extruder building up, because the resin did not feed consistently into the extruder. Similar to the film of Example 1, this film also had sporadic holes in it caused by the presence of tiny unmelted particles.

The films of Examples 3 and 5 were smooth, soft, strong, and flexible. The film of Example 3 contained occasional unmelted particles, which appeared to be TiO₂. The film of Example 5 was not uniform in thickness, since there was slight surging of the melted resin observed. Slight ribboning of one side of the Example 5 film was also observed.

The films of Examples 4 and 6 appeared to be soft and slightly grainy in texture, which was due to small unmelted particles of TiO₂. The un-melted particles led to the presence of small fish-eye holes in the films. The films did not appear to be as strong as the films of Examples 3 and 5, which contained PE instead of EVA. The films were also marbled in appearance, particularly when the thickness was decreased. The marbled appearance was either due to an uneven distribution of the TiO₂ in the resin, or a slight incompatibility between the ETPS, EVA, and the TiO₂.

The film Example 7 was soft and white. As the film was cast thinner, unmelted TiO₂ particles became noticeable and holes began to form. A marbling effect was observed with the coloring of the film as it became thinner. There were also fluctuations in the pressure in the die, which was as low as 5 psi and as high as 1200 psi.

Both films of Example 8 and Example 9 were smooth, soft, flexible, white in color, and had a papery feel to them. The edges of both films had ribboning. The film of Example 8 had occasional small holes in it, which was caused by unmelted particles. The film of Example 9 did not have holes in it until it was brought to a thickness of approximately 1.0-1.2 mil.

The films of Example 10 and Example 11 were both cast in order to determine the maximum temperature films could be cast, and to determine how temperature affects film casting and mechanical properties.

### Testing Mechanical Properties of Films

### Example 13

Films were tested for tensile properties (peak stress, modulus, strain at break, and energy per volume at break) using two different methods. Tensile testing was performed on a Sintech 1/D. Five samples were tested for each film in both the machine direction (MD) and the cross direction (CD). A computer program called TestWorks 4 was used to collect data during testing and to generate a stress versus strain curve from which a number of properties were determined, including modulus, peak stress, elongation, and toughness, which will be addressed in the Results and Discussion section.

The first method of testing was based on ASTM D638-08 Standard Test Method for Tensile Properties of Plastics. Film samples were cut into dog bone shapes with a center width of 3.0 mm before testing. The dog-bone film samples were held in place using grips on the Sintech device with a gauge length of 18.0 mm. The film samples were stretched at a crosshead speed of 5.0 in/min until breakage occurred.

Films were tested for tensile properties based on ASTM D638-08 Standard Testing Method for Tensile Properties of Plastics. Results of this testing are shown in Table 4.

TABLE 4
Film Tensile Properties Using ASTM D638-08 Standard Testing Method for Tensile Properties of Plastics

| | | Average of | | Average of | | Average of | | Average of | | Energy Per | |
|---|---|---|---|---|---|---|---|---|---|---|---|
| | | Thickness | | Peak Stress | | Strain At | | Modulus | | Volume At Break | |
| | | (mil) | | (MPa) | | Break (%) | | (MPa) | | (J/cm³) | |
| Sample ID | Film Composition | MD | CD | MD | CD | MD | CD | MD | CD | MD | CD |
| Comparative | Ecoflex-TPS 60:40 | 1.5 | 1.6 | 12.6 | 8.1 | 440 | 400 | 165 | 116 | 39 | 25 |
| Example 2 | | | | | | | | | | | |
| Comparative | Ecoflex-PE 65:35 | 1.1 | 1.1 | 55.3 | 19.4 | 610 | 600 | 66 | 99 | 159 | 72 |
| Example 3 | | | | | | | | | | | |
| Comparative | Ecoflex | 1.1 | 1.1 | 46.2 | 46.5 | 540 | 850 | 84 | 100 | 144 | 173 |
| Example 5 | | | | | | | | | | | |
| Comparative | PE | 1.0 | 1.0 | 47.6 | 37.7 | 550 | 800 | 70 | 77 | 108 | 137 |
| Example 6 | | | | | | | | | | | |
| Comparative | EVA | 1.3 | 1.2 | 18.8 | 16.7 | 490 | 800 | 18 | 22 | 47 | 60 |
| Example 7 | | | | | | | | | | | |
| Example 1 | EcoflexTPS:PE:Fusabond | 1.1 | 1.1 | 34.4 | 12.1 | 460 | 450 | 104 | 132 | 85 | 40 |
| | (63:34:3) | | | | | | | | | | |
| Example 2 | ETPS:EVA:Fusabond | 1.1 | 1.2 | 13.9 | 8.5 | 470 | 410 | 37 | 69 | 42 | 25 |
| | (6:34:3) | | | | | | | | | | |
| Example 3 | ETPS:PE (65:35) + 2% | 1.2 | 1.1 | 34.9 | 15.2 | 381 | 568 | 61 | 89 | 66 | 52 |
| | TiO2 | | | | | | | | | | |
| Example 4 | ETPS:EVA (65:35) + 2% | 1.3 | 1.3 | 14 | 7.7 | 365 | 419 | 24 | 25 | 32 | 22 |
| | TiO2 | | | | | | | | | | |
| Example 5 | ETPS:PE (60:40) + 2% | 1.1 | 1.1 | 37.3 | 18.2 | 368 | 633 | 66 | 59 | 66 | 63 |
| | TiO2 | | | | | | | | | | |
| Example 6 | ETPS:EVA (60:40) + | 1.3 | 1.3 | 15.4 | 6.4 | 297 | 390 | 20 | 10 | 29 | 16 |
| | 2% TiO2 | | | | | | | | | | |
| Example 7 | Ecoflex:TPS (60:40) + | 1.3 | 1.3 | 13 | 8 | 267 | 397 | 137 | 140 | 26 | 24 |
| | 2% TiO2 | | | | | | | | | | |
| Example 8 | ETPS:PE:Fusabond | 1.3 | 1.3 | 21.8 | 7.7 | 384 | 259 | 66 | 99 | 50 | 16 |
| | (63:34:3) + 2% TiO2 | | | | | | | | | | |
| Example 9 | ETPS:EVA:Fusabond | 1.4 | 1.4 | 11 | 5.7 | 243 | 224 | 27 | 31 | 19 | 10 |
| | (63:34:3) + 2% TiO2 | | | | | | | | | | |
| Example 10 | ETPS:PE:Fusabond | 1.7 | 1.7 | 19.7 | 5.8 | 567 | 181 | 68 | 47 | 60 | 9 |
| | (63:34:3) + 5% Ampacet | | | | | | | | | | |
| Example 11 | ETPS:PE (65:35) + | 1.4 | 1.4 | 21.6 | 7.7 | 535 | 352 | 68 | 58 | 64 | 20 |
| | 5% Ampacet | | | | | | | | | | |

*Example 10 data represents the casting temperature settings of 160, 165, 170, 170, 175° C.
Example 11 data represents the casting temperature settings of 155, 160, 165, 165, 170° C.

The results showed that films made from codes 1 and 2 had balanced mechanical properties. The films were soft and flexible and also had adequate tensile properties.

The second method of tensile testing utilized standard ASTM D882-02. For this method, film samples with a width of 1.0 inches (25.40 mm) and an approximate length of 3.0 inches were prepared. The film samples were held in place using grips on the Sintech device with a gauge length of 50.0 mm. The films were extended at a crosshead speed of 500.00 min/min until breakage occurred. The load limit high was set at 10 kgf. Results of this testing are shown in Table 5.

TABLE 5
Biodegradable Film Tensile Properties Based on ASTM D882-02
Standard Testing Method for Tensile Properties of Plastics

| | | Average | | Average | | Average | | Average | | | |
|---|---|---|---|---|---|---|---|---|---|---|---|
| | | Thickness | | Peak Load | | Peak Stress | | Strain at | | Average | |
| | | (mil) | | (gf) | | (psi) | | Break (%) | | Modulus (psi) | |
| Sample ID | Film Composition | CD | MD | CD | MD | CD | MD | CD | MD | CD | MD |
| Comparative | Ecoflex/TPS (60/40) | 1.5 | 1.5 | 890 | 840 | 1310 | 1250 | 520 | 340 | 29100 | 20900 |
| Example 2 | | | | | | | | | | | |
| Comparative | Ecoflex:PE (65:35) | 1.1 | 1.0 | 760 | 2450 | 1560 | 5480 | 420 | 550 | 14600 | 8400 |
| Example 3 | | | | | | | | | | | |
| Comparative | Ecoflex (100) | 1.2 | 1.3 | 1740 | 1560 | 3180 | 2600 | 690 | 320 | 14900 | 14500 |
| Example 5 | | | | | | | | | | | |
| Comparative | PE (100) | 1.1 | 1.1 | 1340 | 1830 | 2690 | 3860 | 560 | 560 | 15300 | 10000 |
| Example 6 | | | | | | | | | | | |
| Comparative | EVA (100) | 1.4 | 1.3 | 680 | 1000 | 1040 | 1680 | 500 | 410 | 2800 | 2500 |
| Example 7 | | | | | | | | | | | |
| Example 1 | ETPS:PE:Fusabond | 1.0 | 1.1 | 670 | 1900 | 1430 | 3790 | 440 | 490 | 25600 | 15400 |
| | (63:34:3) | | | | | | | | | | |
| Example 2 | ETPS:EVA:Fusabond | 1.4 | 1.3 | 410 | 770 | 660 | 1340 | 220 | 500 | 8100 | 9000 |
| | (63:34:3) | | | | | | | | | | |
| Example 3 | ETPS:PE (65:35) + | 1.1 | 1.0 | 720 | 1840 | 1410 | 3980 | 480 | 400 | 17700 | 11400 |
| | 2% TiO2 | | | | | | | | | | |
| Example 4 | ETPS:EVA (65:35) + | 1.2 | 1.2 | 520 | 970 | 930 | 1810 | 430 | 380 | 8000 | 7500 |
| | 2% TiO₂ | | | | | | | | | | |
| Example 5 | ETPS:PE (60:40) + | 1.1 | 1.0 | 780 | 1850 | 1640 | 4120 | 520 | 360 | 19500 | 12700 |
| | 2% TiO2 | | | | | | | | | | |
| Example 6 | ETPS:EVA (60:40) + | 1.1 | 0.7 | 450 | 920 | 900 | 2810 | 400 | 270 | 7600 | 10800 |
| | 2% TiO₂ | | | | | | | | | | |
| Example 7 | Ecoflex:TPS (60:40) + | 0.8 | 0.7 | 630 | 930 | 1650 | 2900 | 400 | 310 | 31900 | 33700 |
| | 2% TiO₂ | | | | | | | | | | |
| Example 8 | ETPS:PE:Fusabond | 1.2 | 1.2 | 530 | 1360 | 940 | 2530 | 250 | 340 | 18200 | 15500 |
| | (63:34:3) + 2% TiO₂ | | | | | | | | | | |
| Example 9 | ETPS:EVA:Fusabond | 1.4 | 1.4 | 480 | 890 | 750 | 1370 | 300 | 290 | 7300 | 7300 |
| | (63:34:3) + 2% TiO₂ | | | | | | | | | | |

### Example 14

The formulation is the same as in Example 1, except that the ETPS (Comparative Example 2) was made on the same twin screw extruder using a high intensity screw with 17 pairs of kneading screw elements versus 7 pairs of kneading blocks for making ETPS used in Example 1. The added kneading blocks provided increased intensity and level of mixing. The present inventive compositions can avoid gel particles or un-melted particles, which are a defect when they appear as solid particles in the finished films.

The resulting pellets were processed into cast films using a Haake cast film line. The blend pellets were processed into cast films on the same film extrusion equipment as described in Example 12. The process conditions on the HAAKE cast film equipment were:

Temperatures: 140° C., 150° C., 160° C., 160° C., and 150° C.

Melt temperature: 161° C.

Torque: 3600 to 3700 m.g.

It was surprising to find the blend pellets of Example 14 can be processed at much lower temperatures than Example 1 (about 215 to 25° C. lower). The melt temperature was also about 20 to 27° C. lower as well. The film samples made from this improved process had no gels, while the films made from the resin using low intensity mixing screws had some visible gel-like defects. The tensile properties were tested. This improvement allowed the film gauge to be reduced from 1.8 mil to 1.1 mil, resulting in a significant material savings.

The film was tested using ASTM D638-08 Standard Testing Method, the film had tensile peak stress of 42 MPa and 15 MPa in MD and CD; a strain-at-break of 639% and 635% in MD and CD; modulus of 19 MPa and 24 MPa in MD and CD; and energy-at-break of 135 J/cm³ and 54 J/cm³ in MD and CD.

Nonetheless, even with a thinner film, the present Example 14 exhibited better physical properties relative to film samples of Example 1. An improved tensile strength is observed for samples of Example 14 films made from high intensity mixing and contained no gels. In comparing films made from the same composition but using a lower intensity screw as in Example 1, the film made from a high intensity screw (Example 14) had the average MD and CD tensile strengths increased by 22% and 24% respectively over those of the film of Example 1. The film in Example 14 also had 39% and 41% higher elongation-at-break for MD and CD respectively than those in Example 1. The same trend was found for energy-at-break, the film of Example 14 had 59% and 35% higher energy-at-break for MD and CD respectively than those of film of Example 1. The film can be used packaging film for wide variety of products. It can also be used backsheet film for diapers, training pants, and adult incontinence products; as well as the baffle film for feminine and adult incontinence pad and pantiliner.

### Example 15

The polymer blend pellets made in Example 14 was made into a blown film using a HAAKE Rheomex 252 single screw extruder fitted with 1 inch diameter blown film die and cooling tower manufactured by HAAKE. The blown film processing conditions are as follows:

Temperature: 160° C., 170° C., 170° C., 160° C., and 160° C.

Melt Temperature: 142° C.

Torque: 2150 to 2200 m.g.

The blown film was tested using ASTM D638-08 Standard Testing Method. The film had tensile peak stress of 26.7 MPa and 21.0 MPa in MD and CD; strain-at-break of 722% and 690% in MD and CD; modulus of 44 MPa and 55 MPa in MD and CD; and energy-at-break of 100 J/cm³ and 81 J/cm³ in MD and CD respectively. As compared with the cast film of Example 14, the film of Example 15 is more balanced in MD and CD properties. The film can be used packaging film for wide variety of products. It can also be used backsheet film for diapers, training pants, and adult incontinence products; as well as the baffle film for feminine and adult incontinence pad and pantiliner.

### Example 16

Machine direction sections were prepared by fracturing the films in the MD direction after chilling the film samples to a cryogenic temperature in liquid nitrogen. The cross direction sections were prepared by cutting the film in the cross-direction using a cryogenically chilled SUPER-KEEN razor while the sample was maintained at cryogenic temperature. The sections were mounted vertically and sputter coated with gold using light burst applications at low current to significantly reduce any possibility of sample heating.

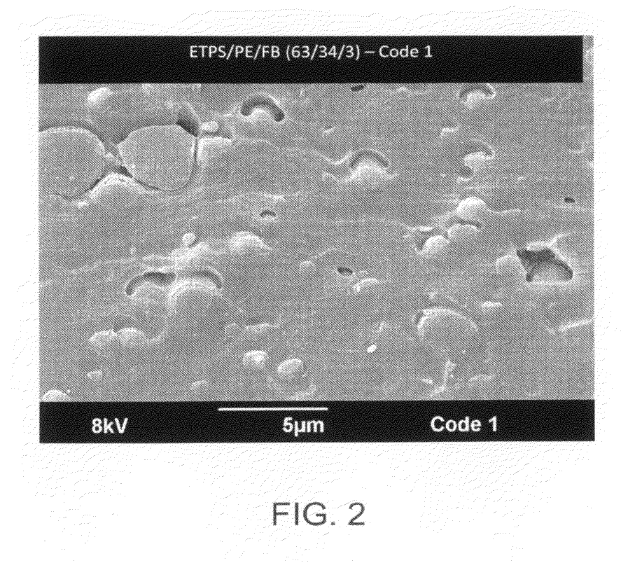

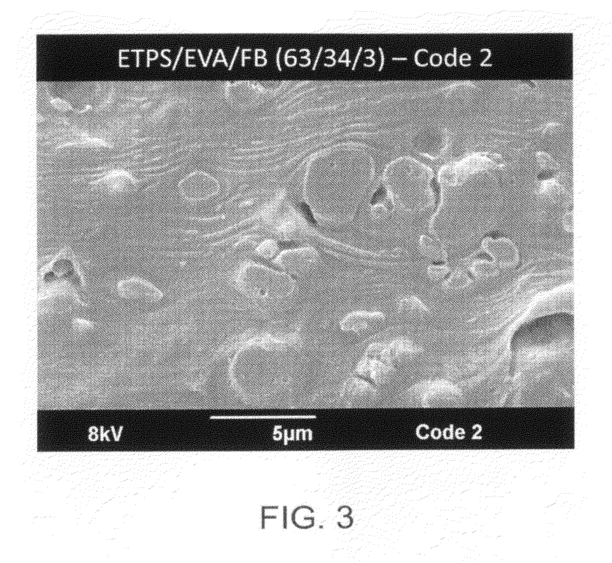

All samples were examined using a JEOL 6490LV scanning electron microscope (SEM) operated at low voltage. FIG. 2 is a SEM image of a cross-section of a film of Example 1. FIG. 3 is a SEM image of the cross section of Example 2. Both images showed the films of the invention had multiple phases compatibilized in the blend. With the presence of various sized micro-structured dispersed phases, it was surprising that the resulting films had the observed excellent mechanical properties.

The present invention has been described in general and in detail by way of examples. Persons of skill in the art understand that the invention is not limited necessarily to the embodiments specifically disclosed, but that modifications and variations may be made without departing from the scope of the invention as defined by the following claims or their equivalents, including other equivalent components presently known, or to be developed, which may be used within the scope of the present invention. Therefore, unless changes otherwise depart from the scope of the invention, the changes should be construed as being included herein.

We claim:

1. A thin thermoplastic film prepared from a polymer blend system comprising: about 5 wt. % to about 40 wt. % of a polyolefin, about 5 wt. % to about 45 wt. % of a plasticized natural polymer, about 5 wt. % to about 75 wt. % of a biodegradable polymer, and about 0.5 wt. % to about 15 wt. % of a compatibilizer having both a polar and a non-polar moiety on the same polymer molecule, where total amount of natural and biodegradable components in said cast film constitute a majority phase of at least 53 wt. % of dry polymer blend.

2. The thermoplastic film according to claim 1, wherein said polymer blend system is substantially absent gel particles.

3. The thermoplastic film according to claim 1, wherein said total biodegradable components in said cast film constitutes at least 55 wt. % of polymer blend.

4. The thermoplastic film according to claim 1, wherein the amount by weight of each of the component classes may range as follows: polyolefin from about 7% to about 30%; plasticized natural polymers from about 5% to about 35%; biodegradable polymer from about 15% to about 65%; and compatibilizer from about 0.5% to about 12.5%.

5. The thermoplastic film according to claim 1, wherein said plasticized natural polymer is a thermoplastic starch, a thermoplastic plant protein, thermoplastic algae.

6. The thermoplastic film according to claim 1, wherein said biodegradable polymer is an aliphatic-aromatic copolyester, polycaprolactone, polyesteramides, modified polyethylene terephthalate, polylactic acid (PLA) and its copolymers, terpolymers based on polylactic acid, polyglycolic acid, polyalkylene carbonates (such as polyethylene carbonate), polyhydroxyalkanoates (PHA), poly-3-hydroxybutyrate (PHB), poly-3-hydroxyvalerate (PHV), poly-3-hydroxybutyrate-co-4-hydroybutyrate, poly-3-hydroxybutyrate-co-3-hydroxyvalerate copolymers (PHBV), poly-3-hydroxybutyrate-co-3-hydroxyhexanoate, poly-3-hydroxybutyrate-co-3-hydroxyoctanoate, poly-3-hydroxybutyrate-co-3-hydroxydecanoate, poly-3-hydroxybutyrate-co-3-hydroxyoctadecanoate, polybutylene succinate, polybutylene succinate adipate, polyethylene succinate.

7. The thermoplastic film according to claim 1, wherein said polyolefin is polyethylene, polypropylene, copolymer of ethylene and propylene, polyethylene-co-vinyl acetate, and mixture of tow and more polyolefins.

8. The thermoplastic film according to claim 1, wherein said compatibilizer is a polar monomer-grafted polyolefin.

9. The thermoplastic film according to claim 1, wherein said compatibilizer is a copolymer of at least one polar monomer and one or more olefinic monomers.

10. The thermoplastic film according to claim 1, wherein said compatibilizer is one of the following: a maleic anhydride, acrylic acid, glycidyl acrylate, glycidyl methacrylate, glycidyl acrylate, or other polar monomer grafted polyolefins.

11. The thermoplastic film according to claim 1, wherein said the thin film has a thickness from about 10 micrometers to about 40 micrometers.

12. The thermoplastic film according to claim 1, wherein said film has a compatibilized microstructure with finely dispersed minority component.

13. The thermoplastic film according to claim 1, wherein said film has a continuous phase of biodegradable polymers.

14. The thermoplastic film according to claim 1, wherein said film has a dispersed thermoplastic starch.

15. The thermoplastic film according to claim 1, wherein said film has a peak stress of at least 21 MPa in MD and 7 Mpa in CD.

16. The thermoplastic film according to claim 1, wherein said film has a strain-at-break at least 600% in MD and about 300% in CD.

17. The thermoplastic film according to claim 1, wherein said film has an energy-at-break of at least 70 Joules per cubic centimeter in MD and lat least 18 Joules per centimeter in CD.

18. The thermoplastic film according to claim 1, wherein said film also includes a pigment, an antioxidant, slips additives, and anti-blocking agents.

19. The thermoplastic film according to claim 1, wherein said pigment, antioxidant, slip additives, and anti-blocking agents, etc, are up to about 5 or 6 wt. % total.

20. An absorbent consumer article comprising: a top sheet, a back sheet, an absorbent core situated between said top sheet and back sheet said back sheet comprising a film formed from a polymer blend having a plasticized natural polymer, a biodegradable polymer, a polyolefin, and a compatibilizer with both a polar and a non-polar moiety on the same polymer molecule, where total biodegradable components in said cast film constitute at a majority phase of least 53 wt. % of dry polymer blend.

21. The absorbent consumer product according to claim 20, wherein said product is: a diaper, an adult incontinence article, a feminine hygiene product, and other product for hygiene absorbent uses.

22. A method of forming a film, the method comprising: providing a polymer blend including a plasticized natural polymer, a biodegradable polymer, a polyolefin, and a compatibilizer with both a polar and a non-polar moiety on the same polymer molecule, where total biodegradable components in said cast film constitute at a majority phase of least 53 wt. % of dry polymer blend; mixing said polymer blend under melt extrusion conditions; extruding said polymer blend, and forming a film sheet.
